SEQUENCE LISTING (8 sequences)

SEQ ID NO: 1 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 2 - protein, 37 aa, Homo sapiens
AAKGTGITRKKTFKEVANAVKISASLMGTVMAKRVKA

SEQ ID NO: 3 - protein, 37 aa, Bos taurus
ATKGAGITRKKTFKEVANAVKISASLMGTLMAKRVKA

SEQ ID NO: 4 - protein, 38 aa, Homo sapiens
GTNGTPTKRRAIGFKKLAEAVKFSAKLMGQAMAKRVKA

SEQ ID NO: 5 - protein, 38 aa, Rattus rattus; identical to SEQ ID NO: 4

SEQ ID NO: 6 - protein, 37 aa, Rattus rattus
DEKLRPRRREIRFTVLVKAVFFASVLMRKVMASRVRA

SEQ ID NO: 7 - protein, 37 aa, Mus musculus
NEKLRPRRREIRFRVLVKVVFFASMLMRKVMASRVRA

SEQ ID NO: 8 - protein, 37 aa, Homo sapiens
DEKRRPKRREIPLKVLVKAVLFACMLMRKTMASRVRV

(19) United States
(12) Patent Application Publication
(10) Pub. No.: US 2004/0120930 A1
(43) Pub. Date: Jun. 24, 2004

(54) Gene therapy for critical limb ischemia with wild type or mutant eNOS

(75) Inventors: William P. Dole, Alamo, CA (US); Katalin Kauser, El Sobrante, CA (US); Hu Sheng Qian, Walnut Creek, CA (US); Gabor Rubanyi, Alamo, CA (US)

Correspondence Address: BERLEX BIOSCIENCES, PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/642,255

(22) Filed: Aug. 15, 2003

Related U.S. Application Data
(60) Provisional application No. 60/403,637, filed on Aug. 16, 2002.

(51) Int. Cl. A61K 48/00

(52) U.S. Cl. 424/93.2; 514/44

(57) ABSTRACT

The present invention provides novel methods of preventing, diagnosing, and treating Critical Limb Ischemia (CLI), using eNOS polypeptides and polynucleotides to modulate eNOS activity in cells. Wild-type and mutant eNOS polypeptides, and polynucleotides encoding such polypeptides, are provided for use in the methods of the present invention. The eNOS mutant polypeptides of the present invention have at least one mutation corresponding to a site in a functional domain of a mammalian eNOS that is phosphorylated in cells.

This application claims the benefit of U.S. Provisional Application Serial No. 60/403,637, filed on Aug. 16, 2002, which is incorporated herein by reference.

### FIELD OF THE INVENTION

The present invention relates to methods of preventing, diagnosing, or treating Critical Limb Ischemia (CLI), using eNOS polypeptides and polynucleotides to modulate eNOS activity in cells. Wild-type and mutant eNOS polypeptides, and polynucleotides encoding such polypeptides, are provided for use in the methods of the present invention.

### BACKGROUND OF THE INVENTION

The prevalence of peripheral arterial occlusive disease (PAOD) in the US population is approximately 12%. Early PAOD (claudication) is characterized by ischemic muscle pain with exercise. Critical limb ischemia (CLI), due to advanced PAOD, is characterized by reduced blood flow and oxygen delivery at rest, resulting in muscle pain at rest and non-healing skin ulcers or gangrene (Rissanen et al., Eur. J. Clin. Invest 31:651-666 (2001); Dormandy and Rutherford, J. Vasc. Surg. 31:S1-S296 (2000)) and is estimated to effect at least 500-1000 per million per year.

The treatment of patients with PAOD and claudication includes the control of atherosclerotic risk factors, exercise and in some cases medical therapy with Cilastozol or Pentoxifylline (Robeer et al., Eur. J. Vasc. Endovasc. Surg. 15:36-43 (1998). If symptoms persist, percutaneous transluminal angioplasty (PTA) may be used for the treatment of claudication, rest pain and/or nonhealing ischemic ulcers. PTA is best suited for short stenosis or occlusions of the iliac and superficial femoral arteries. Patency rates at 1 year are 80-90%. In patients with more diffuse disease, surgical revascularization is recommended. Aortofemoral and femoropopliteal bypass grafting have 5 year patency rates of 90% and 70%, respectively (Dormandy and Rutherford, J. Vasc. Surg. 31:S1-S296 (2000).

However, despite advances in surgical and interventional revascularization techniques, 20-30% of patients presenting with ischemic pain and ulcers are either not suitable candidates for surgical revascularization or angioplasty due to diffuse, distal vascular disease or fail such therapy, and current pharmacotherapy has had little impact on limb salvage in patients with advanced CLI.

The survival rate of such patients without major amputation is less than 50% after 1 year. Furthermore, older patients who require amputation frequently have poor general health and have relatively high operative risk and poor long term prognosis (Dormandy and Rutherford, J. Vasc. Surg. 31:S1-S296 (2000). While angiotensin-converting enzyme inhibitors and statins have been reported to augment blood flow in experimental models of ischemia, none of the currently available drugs promote new blood vessel formation (Fabre et al., Circulation 99:3043-3049 (1999); Kureishi et al., Nat Med 6:1004-1010 (2000). There is, therefore, a need for improved therapeutic approaches to CLI.

One new approach for treating ischemic disorders is to target the enhancement of blood vessel growth using growth factors that stimulate vessel formation (Rissanen et al., Eur. J. Clin. Invest. 31:651-666 (2001); Yla-Herttuala et al., Lancet 355:213-222 (2000); Isner et al. J. Clin. Invest. 103:1231-1236 (1999); Rutanen et al., Curr. Cardiol. Report 3:29-36 (2001)). Vascular growth can be divided into vasculogenesis, angiogenesis and arteriogenesis. Vasculogenesis refers to embryonic in situ formation of blood vessels from angioblasts/endothelial precursor cells (EPCs). Angiogenesis refers to formation of new blood vessels from preexisting capillaries, as a result of proliferation and migration of differentiated endothelial cells (ECs) (Risau et al., Nature 386: 671-674 (1997)). Arteriogenesis refers to in situ formation of muscular collateral vessels from preexisting arteriolar anastomoses (Schaper et al., Circ Res 79:911-919 (1996). However, endogenous angiogenesis and arteriogenesis are inadequate to fully compensate for the reduction in blood flow and oxygen delivery in CLI. Although the delivery of growth factors to stimulate blood vessel formation may potentially be an additional therapeutic approach for treatment of CLI, there is growing evidence that the impairment of endothelial function with age, atherosclerosis and other cardiovascular risk factors may limit augmentation of angiogenesis with growth factors.

Endothelial nitric synthase (eNOS, also called ecNOS or NOS 3) has been implicated as an important regulator/mediator of angiogenesis. Nitric oxide (NO) donors, such as nitroprusside, promote endothelial cell proliferation and migration, whereas NOS inhibitors suppress these processes (Ziche et al., J. Clin. Invest. 9:2036-2044 (1994); Morbidelli et al., Am. J. Physiol. 270:H411-H415 (1996)). Studies have shown impaired angiogenesis and wound healing in mice deficient in eNOS (eNOS-KO). Using explanted aortas, Lee et al. demonstrated that eNOS is required for endothelial cell migration, proliferation and differentiation in vitro. This finding was confirmed in vivo, by demonstrating reduced capillary formation into subcutaneously implanted Matrigel plugs in eNOS-KO mice. Excisional wound healing was also significantly delayed in eNOS-KO mice emphasizing the role of endothelial NO in the process of angiogenesis associated with wound repair (Lee et al., Am. J. Physiol. 277:H1600-H1608 (1999)). Significantly impaired angiogenesis has been demonstrated in eNOS-KO mice following surgically induced hindlimb ischemia. L-arginine (the substrate of NOS) administration to rabbits with surgical hindlimb ischemia, significantly improved angiogenesis, confirming the role of endothelial NO in ischemia-induced angiogenesis (Murohara et al., J. Clin. Invest. 101:2567-2578 (1998)).

As mentioned, there is also increasing evidence that angiogenesis in response to ischemia may be impaired with age and endothelial dysfunction. Impaired angiogenesis has been observed in animal models, likely resulting from reduced release of endothelial NO and diminished expression of growth factors (Rivard et al., Circulation 99:111-120 (1999); Van Belle et al., Circulation 96:2667-2674 (1997)). Risk factors, such as homocysteinemia and hypercholesterolemia have been shown to attenuate angiogenesis in a rat model of hindlimb ischemia possibly by decreasing the bioavailability of endothelial derived NO (Duan et al., Circulation 102:111370-111376 (2000)).

In view of the above, there is a need for an effective treatment for CLI. The development of a gene therapy approach for the treatment of CLI which increases NO bioavailability in an ischemic limb may reverse ischemia through multiple mechanisms, including e.g., by stimulating impaired angiogenesis, ameliorating existing microvascular dysfunction, restoring vasomotor (vasodilator) activity in existing vessel, and contributing to the remodeling/maturation of existing collaterals (arteriogenesis).

### SUMMARY OF THE INVENTION

The present invention provides methods of preventing, diagnosing, and treating CLI, using eNOS polypeptides or polynucleotides encoding such polypeptides. Using methods of the present invention, eNOS activity can be modulated in cells such that a disease or disorder associated with eNOS activity can be ameliorated. In particular, using the methods of the present invention, eNOS activity can be modulated in cells by increasing local NO production in an ischemic limb such that a disease or disorder associated with reduced NO production in an ischemic limb can be ameliorated. Thus, the invention provides methods of gene therapy which provide effected tissues with increased levels of NO, through administration of an eNOS polypeptide or mutant thereof, or polynucleotide encoding such polypeptides, to a patient in need of treatment.

In one aspect, the invention provides a method of treating CLI comprising administering to a patient in need of treatment an effective amount of a polynucleotide encoding a mammalian eNOS polypeptide.

The invention further provides methods of treating symptoms of CLI, e.g., microvascular dysfunction, ulcer healing, and angiogenesis. In one aspect, the invention provides a method of treating angiogenesis comprising administering to a patient in need of treatment an effective amount of a polynucleotide encoding an eNOS polypeptide, wherein the eNOS polypeptide comprises at least one mutation at a position corresponding to an amino acid residue in a mammalian eNOS that is phosphorylated in mammalian cells. In one aspect, the invention provides a method of ameliorating microvascular dysfunction comprising administering to a patient in need of treatment an effective amount of a polynucleotide encoding an eNOS polypeptide, wherein the eNOS polypeptide comprises at least one mutation at a position corresponding to an amino acid residue in a mammalian eNOS that is phosphorylated in mammalian cells.

In a related aspect, the present invention provides eNOS polypeptides, and polynucleotides encoding eNOS polypeptides, for use in the methods of the present invention. Suitable polynucleotides for use in the methods of the present invention include, e.g., polynucleotides encoding a wild-type or mutant eNOS polypeptide, or variants thereof. Preferably, the encoded eNOS polypeptide is a mammalian eNOS polypeptide, and most preferably a human eNOS polypeptide. In one aspect, the eNOS polypeptide is the human eNOS polypeptide encoded by SEQ ID NO: 1.

In another aspect, the human eNOS polypeptide encoded by a polynucleotide suitable for use in the methods of the present invention has a first mutation at a position corresponding to amino acid residue 495 of SEQ ID NO: 1; and a second mutation at a position corresponding to amino acid residue 1177 of SEQ ID NO: 1.

In another aspect, the human eNOS polypeptide encoded by a polynucleotide suitable for use in the methods of the present invention, has a first mutation at a position corresponding to amino acid residue 495 of SEQ ID NO: 1; a second mutation at a position corresponding to amino acid residue 1177 of SEQ ID NO: 1; and a third mutation at a position corresponding to amino acid residue 2 of SEQ ID NO: 1.

Preferably, the mutation at a position corresponding to amino acid residue 495 of SEQ ID NO: 1 is an amino acid substitution to Ala, Gly, Val, Leu, Ile, Pro, Phe, Tyr, Trp, Met, Ser, Cys, Glu, Asn, Gln, Lys, Arg, or His and is more preferably Ala, Val, Leu, or Ile, and most preferably Ala or Val; the mutation corresponding to amino acid residue 1177 of SEQ ID NO: 1 is an amino acid substitution to Asp; and the mutation at a position corresponding to amino acid residue 2 of SEQ ID NO: 1 is an amino acid substitution to Ala.

In some aspects, the phosphorylation of a human eNOS polypeptide encoded by a polynucleotide suitable for use in the methods of the present invention, is increased or decreased, as compared to a reference polypeptide.

In some aspects, the human eNOS polypeptide encoded by a polynucleotide suitable for use in the methods of the present invention has an increased eNOS activity, as compared to a reference eNOS polypeptide. Preferably, the human eNOS polypeptide has increased NO production, binding affinity for calmodulin, and/or reductase activity, as compared to a reference eNOS polypeptide. Most preferably, the human eNOS polypeptide has increased NO production, as compared to a reference eNOS polypeptide.

In some aspects, the human eNOS polypeptide encoded by a polynucleotide suitable for use in the methods of the present invention has a decreased eNOS activity, as compared to a reference eNOS polypeptide, e.g., decreased Ca++ dependence in Ca++-calmodulin mediated stimulation of the polypeptide, as compared to a reference eNOS polypeptide.

In one aspect, a polynucleotide suitable for use in the methods of the invention encodes an eNOS polypeptide that is substantially homologous to the amino acid of a human eNOS polypeptide.

In one aspect, a polynucleotide suitable for use in the methods of the invention encodes an eNOS polypeptide that has 95-99% sequence identity to the amino acid sequence of SEQ ID NO: 1.

In another aspect, a polynucleotide suitable for use in the methods of the present invention is a recombinant vector encoding a nucleic acid sequence encoding an eNOS polypeptide. In one aspect, the nucleic acid sequence is operably linked to at least one regulatory sequence such that the encoded eNOS polypeptide is expressed in cells. Preferably, at least one regulatory sequence is a promoter, e.g., a CMV promoter. In one aspect, the recombinant vector is a plasmid vector or an adenoviral vector.

In another aspect, in the methods of treatment of the present invention, the treatment is by modulating eNOS activity in the cells of a patient in need of treatment. Preferably, the cells are endothelial cells, and more preferably human endothelial cells.

In another aspect, the methods of the invention further comprise administering one or more angiogenic factors before, during, or after administration to a patient in need of treatment, a polynucleotide encoding a human wild-type or mutant eNOS polypeptide. Suitable angiogenic factors for use in the methods of the present invention include, but are not limited to, e.g., HFG, VEGF, FGF, Endothelial Growth Factor, Epidermal Growth Factor, Platelet-Derived Growth Factor, TGF-alpha, TGF-beta, PDGF, TNA-alpha or IGF, Del-1, and is preferably FGF.

In one aspect, the polynucleotides suitable for use in the methods of the present invention, are administered by introducing the polynucleotides to cells of a patient, ex vivo. In another aspect, the polynucleotides suitable for use in the methods of the present invention, are administered by introducing the polynucleotides to a diseased tissue of a patient, or to the peripheral vascular system of a patient. In another aspect, the polynucleotides suitable for use in the methods of the present invention, are administered by intramuscular injection or intraarterial injection to a limb muscle of the patient.

In an another aspect, the invention provides a method of treating critical limb ischemia (CLI) comprising administering to a patient in need of treatment an effective amount of an eNOS polypeptide, wherein the eNOS polypeptide comprises at least one mutation at a position corresponding to an amino acid residue in a mammalian eNOS that is phosphorylated in mammalian cells. Preferably, the eNOS polypeptide suitable for use in the methods of the present invention is a human eNOS and has at least one mutation at a position corresponding to amino acid residue 495 or 1177 of SEQ ID NO: 1.

In one aspect, the eNOS polypeptide suitable for use in the methods of the present invention has at least one mutation at a position corresponding to amino acid residue 495 or 1177 of SEQ ID NO: 1.

In one aspect, the eNOS polypeptide suitable for use in the methods of the present invention has a first mutation at a position corresponding to amino acid residue 495 of SEQ ID NO: 1; and a second mutation at a position corresponding to amino acid residue 1177.

In one aspect, the eNOS polypeptide suitable for use in the methods of the invention has a first mutation at a position corresponding to amino acid residue 495 of SEQ ID NO: 1; a second mutation at a position corresponding to amino acid residue 1177 of SEQ ID NO: 1; and a third mutation at a position corresponding to amino acid residue 2 of SEQ ID NO: 1.

Preferably, the mutation at a position corresponding to amino acid residue 495 of SEQ ID NO: 1 is an amino acid substitution to Ala, Gly, Val, Leu, Ile, Pro, Phe, Tyr, Trp, Met, Ser, Cys, Glu, Asn, Gln, Lys, Arg, or His and is more preferably Ala, Val, Leu, or Ile, and most preferably Ala or Val; the mutation corresponding to amino acid residue 1177 of SEQ ID NO is an amino acid substitution to Asp: 1; and the mutation at a position corresponding to amino acid residue 2 of SEQ ID NO: 1 is an amino acid substitution to Ala.

### BRIEF DESCRIPTION OF THE DRAWINGS

The foregoing and other objects of the present invention, the various features thereof, as well as the invention itself may be more fully understood from the following description, when read together with the accompanying drawings in which:

FIG. 1 is a diagram illustrating various functional domains of mammalian eNOS. The functional domains include, but are not limited to, e.g., (proceeding from the N-terminus to the C-terminus) a consensus site for myristoylation; two sites for palmitoylation; an oxidase domain; a calmodulin binding site (e.g., amino acids 494-517 of human eNOS), which comprises a consensus sequence for phosphorylation (of e.g., Thr-495 of human eNOS); and a reductase domain. Functional domains of a human eNOS polypeptide also include, e.g., an autoinhibitory loop and a heme-binding, site.

FIG. 2 is a histogram illustrating the stimulation of NO production in HEK 293 cells by eNOS polypeptide mutants having a single or double mutation, as compared with the wild-type human eNOS encoded by SEQ ID NO: 1 (WT). The eNOS polypeptide mutants having a single mutation, have an amino acid substitution to Asp (T495D), Ala (T495A), or Val (T495V) at a position corresponding to Thr-495 of the human eNOS encoded by SEQ ID NO: 1. The eNOS polypeptide mutants having a double mutation, have a first amino acid substitution to Asp at a position corresponding to Ser-1177, and a second amino acid substitution to Asp (T495D+S1177D), Ala (T495AV+S1177D), or Val (T495V+S1177D) at a position corresponding to Thr-495 of the human eNOS encoded by SEQ ID NO: 1.

FIG. 3 is a histogram illustrating the stimulation of NO production in human aortic endothelium cells (HAEC) by eNOS polypeptide mutants having a single mutation, as compared with the wild-type human eNOS encoded by SEQ ID NO: 1 (wild-type). The eNOS polypeptide mutants having a single mutation, have an amino acid substitution to Asp (T495D), Ala (T495A), or Val (T495V) at a position corresponding to Thr-495 of the human eNOS encoded by SEQ ID NO: 1.

FIG. 4 is laser Doppler images and schematic illustration of limb perfusion in mice deficient in wild-type eNOS (ecNOS-KO), as compared to mice having wild-type eNOS (C57BU6), at day 0, 1, 4, 7, 10, 14, 21, and 28 following operation.

FIG. 5 is photographs illustrating gross pathological changes in the limbs of mice deficient in wild-type eNOS (ecNOS-KO), as compared to mice having wild-type eNOS (C57BL/6), at day 0 and 4 following operation.

FIG. 6 is a histogram illustrating the quantitative measurement of collateral formation in the limbs of mice deficient in wild-type eNOS (ecNOS-KO), as compared to mice having a wild-type eNOS (C57B1/6), at day 10 following operation.

FIG. 7 is diagrams illustrating different surgical procedures on the limbs of mice deficient in wild-type eNOS (ecNOS-KO) (top row); photographs illustrating gross pathological changes in the limbs following operation (middle row); and laser Doppler images illustrating perfusion in the limbs following operation (bottom row).

FIG. 8 is a histogram illustrating the effect of different age on spontaneous blood flow recovery in the limbs of 3, 6, and 12 month old mice deficient in wild-type eNOS (ecNOS-KO), as compared to mice having a wild-type eNOS (C57BL/6), at day 0, 1, 3, 7, 17, 21, and 24 following operation.

FIG. 9 is a photographs illustrating the effect of different age on ischemic damage in the limbs of 3, 6, and 11-12 month old mice deficient in wild-type eNOS (ecNOS-KO), at day 10 following operation.

FIG. 10 is a histogram illustrating the transfection efficiency of different intramuscular gene delivery methods by detecting the expression level of luciferase: 1) from a plasmid encoding luciferase delivered by injection of the plasmid pLuc alone (pLuc), delivered by injection of pLuc with electroporation (pLuc+EP) and, additionally, with hyaluronidase pretreatment (pLuc+EP+Hyal); as compared to 2) infection by an adenovirus encoding luciferase (Ad5.1Luc).

FIG. 11 is a histogram illustrating the effect of hind limb ischemia on the transfection efficiency of a plasmid vector, pLuc, encoding luciferase. The plasmid vector was injected into the adductor muscle of C57BL/6 mice and transfection efficiency was determined by measuring the level of gene expression: before operation and pretreatment with the plasmid vector (PT); the same day as the operation (SDT); and at day 3 and 7 following operation.

FIG. 12 is a Western blot and histogram of ELISA illustrating eNOS expression following eNOS gene delivery, to mice deficient in wild-type eNOS (ecNOS-KO), of: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector encoding a wild-type eNOS.

FIG. 13 is photographs illustrating the effect of eNOS gene therapy on limb salvage in 6 month old mice deficient in wild-type eNOS (ecNOS-KO), on day 28 following operation, using: 1) a plasmid vector (pNOS224) encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull).

FIG. 14 is a schematic illustration of the effect of eNOS gene therapy, as measured by the ischemic to normal blood perfusion ratio, on 6 month old mice deficient in wild-type eNOS (ecNOS-KO), before operation (BOP) and at day 0, 1, 4, 7, 10, 14, 18, 21, and 28 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull).

FIG. 15 is a histogram illustrating the effect of eNOS gene therapy, as measured by limb and muscle volume and the amount of replacement fat and inflammatory infiltrates, in 6 month old mice deficient in wild-type eNOS (ecNOS-KO), at day 28 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull), and untreated contralateral leg (limb).

FIG. 16 is photographs illustrating the effect of eNOS gene therapy on the healing of ulcers in 6 month old mice deficient in wild-type eNOS (ecNOS-KO), on day 28 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull).

FIG. 17 is photographs illustrating the effect of eNOS gene therapy on limb salvage, in 11-12 month old mice deficient in wild-type eNOS (ecNOS-KO), at day 10 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull).

FIG. 18 is a histogram of eNOS expression (top row); laser Doppler images of blood flow (middle row); and photographs of limb necrosis (bottom row), illustrating the effect of eNOS gene therapy in 11-12 month old mice deficient in wild-type eNOS (ecNOS-KO), at day 10 following operation, using a plasmid vector, pNOS224, encoding an eNOS polypeptide mutant (ecNOS) having an amino acid substitution to Asp (S1177D) at a position corresponding to Ser-1177 of a human eNOS encoded by SEQ ID NO: 1. Gene expression of pNOS224 correllated with limb necrosis and changes in flow, i.e., the higher the expression (#1205) the higher the flow, and smaller the necrotic damage, compared to a lower expressor (#1201).

FIG. 19 is a schematic illustration of the effect of eNOS gene therapy on limb salvage, in 11-12 month old mice deficient in wild-type eNOS (ecNOS-KO), at day 1, 3, 7, and 10 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull).

FIG. 20 is a schematic illustration of the effect of eNOS gene therapy, as measured by the ischemic to normal blood perfusion ratio, in 11-12 month old mice deficient in wild-type eNOS (ecNOS-KO), at day 1, 3, 7, and 10 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull).

FIG. 21 is a histogram illustrating the level of eNOS expression in the adductor muscle of 11-12 month old mice deficient in wild-type eNOS (ecNOS-KO), at day 10 following operation, using: 1) a plasmid vector (pNOS224), encoding an eNOS polypeptide mutant having an amino acid substitution to Asp (S1177D) at a position corresponding to Ser-1177 of a human eNOS encoded by SEQ ID NO: 1; as compared to 2) a plasmid vector not encoding an eNOS or empty vector (pNull), and 3) control unoperated ecNOS-KO mice. The right side of the histogram illustrates individual expression levels from the treated animals.

FIG. 22 is a schematic representation and photographs illustrating a rat CLI model. The schematic representation illustrates the surgical ligation and removal of the femoral artery and branches, supplying the upper thigh area in developed male adult Sprague-Dawley rats. The photographs illustrate gross pathological changes in the rat CLI model at day 1, 4, 10, and 17 following operation.

FIG. 23 is an image of rat CLI angiography illustrating a normal limb (left side of image) and an ischemic limb (right side of image) of the rat CLI model.

FIG. 24 is an image of rat CLI angiography illustrating the angiographic scoring of arteriogenesis in a normal limb (left side of image) and an ischemic limb (right side of image) of the rat CLI model.

FIG. 25 is a schematic illustration of blood flow recovery in the rat CLI model following eNOS gene therapy using adenovirus vector encoding an eNOS polypeptide mutant having an amino acid substitution at a position corresponding to Ser-1177 of the human eNOS encoded by SEQ ID NO: 1 (Ad5NOS1177D); as compared to a control adenovirus vector encoding a green fluorescent protein (Ad5EGFP), before operation (BO) and at day 0, 3, 7, and 10 after gene delivery.

FIG. 26 is a diagram and photographs illustrating the necrotic score based on gross pathological Stages I-V used to measure the effect of gene therapy in the rat CLI model using adenovirus vector encoding an eNOS polypeptide mutant having an amino acid substitution at a position corresponding to Ser-1177 of the human eNOS encoded by SEQ ID NO: 1 (Ad5NOS1177D).

FIG. 27 is a histogram illustrating the necrotic score based on gross pathological Stages I-V, used to measure the effect of gene therapy in the rat CLI model using adenovirus vector encoding an eNOS polypeptide mutant having an amino acid substitution at a position corresponding to Ser-1177 of the human eNOS encoded by SEQ ID NO: 1 (Ad5NOS1177D); as compared to a control adenovirus vector encoding a green fluorescent protein (Ad5EGFP), before operation (BO) and at day 3, 7, and 10 after gene delivery.

FIG. 28 is a histogram illustrating the angiographic score based on measurement of arteriogenesis, used to measure the effect of gene therapy in the rat CLI model using adenovirus vector encoding an eNOS polypeptide mutant having an amino acid substitution at a position corresponding to Ser-1177 of the human eNOS encoded by SEQ ID NO: 1 (Ad5NOS1177D) ; as compared to a control adenovirus vector encoding a green fluorescent protein (Ad5EGFP), at the end of the experiment.

### DETAILED DESCRIPTION OF THE INVENTION

The present invention provides a novel approach for the treatment of Critical Limb Ischemia (CLI). More specifically, the present invention provides methods of preventing, diagnosing, and treating CLI, using eNOS polypeptides or polynucleotides. Using the methods of the present invention, eNOS activity can be modulated in cells such that a disease or disorder associated with eNOS activity can be ameliorated. In particular, using the methods of the present invention, eNOS activity can be modulated in cells by increasing local NO production in an ischemic limb such that a disease or disorder associated with reduced NO production in an ischemic limb can be ameliorated. Thus, the compositions and methods of the present invention provide a novel therapeutic approach for providing increased levels of NO in diseased tissues, thereby, targeting the underlying pathophysiology of CLI through multiple mechanisms including, e.g.,: 1) the stimulation of angiogenesis; 2) the amelioration of microvascular dysfunction; 3) the restoration of vasomotor (vasodilator) activity of existing vessels, and 4) the remodeling/maturation of existing collaterals (arteriogenesis), that are known to be ameliorated by increased NO levels. The resulting improvement of blood flow and oxygen delivery to both skin and muscle is expected to relieve rest pain and heal ischemic ulcers. Moreover, the eNOS polypeptide mutants of the present invention can be more efficacious than wild-type eNOS, due to the significantly higher specific activity of the mutant eNOS enzyme. In addition, the activity of eNOS polypeptides can be tightly regulated by calcium, resistant to oxidized low-density lipoprotein (oxLDL), and age. Consequently, in contrast to growth factors, toxicity due to “overdosing” could be negligible using the eNOS compositions of the present invention in gene therapy applications.

The references cited herein are incorporated by reference, in their entirety.

Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Standard reference works setting forth the general principles of recombinant DNA technology include Sambrook, J., et al. (1989) Molecular Cloning,: A Laboratory Manual, 2d Ed., Cold Spring Harbor Laboratory Press, Planview, N.Y.; McPherson, M. J., Ed. (1991) Directed Mutagenesis: A Practical Approach, IRL Press, Oxford; Jones, J. (1992) Amino Acid and Peptide Synthesis, Oxford Science Publications, Oxford; Austen, B. M. and Westwood, O. M. R. (1991) Protein Targeting and Secretion, IRL Press, Oxford. Any suitable materials and/or methods known to those of ordinary skill in the art can be utilized in carrying out the present invention; however, preferred materials and/or methods are described. Materials, reagents and the like to which reference is made in the following description and examples are obtainable from commercial sources, unless otherwise noted.

As used herein, “polypeptide” refers to a full-length protein or fragment thereof, or peptide. In preferred embodiments, an eNOS fragment or peptide of the present invention retains an eNOS activity, e.g., NO production. However, the level of eNOS activity may be increased or decreased as compared to a reference eNOS polypeptide (e.g., as compared to a full-length or wild-type polypeptide).

As used herein, “variant” with reference to a polypeptide or polynucleotide, refers to a polypeptide or polynucleotide that may vary in primary, secondary, or tertiary structure, as compared to a reference polypeptide or polynucleotide, respectively (e.g., as compared to a wild-type polypeptide or polynucleotide). For example, the amino acid or nucleic acid sequence may contain a mutation or modification that differs from a reference amino acid or nucleic acid sequence. In some embodiments, an eNOS variant may be a different isoform or polymorphism. Variants can be naturally-occurring, synthetic, recombinant, or chemically modified polypeptides or polynucleotides isolated or generated using methods well known in the art.

As used herein, “mutation” with reference to a polypeptide or polynucleotide, refers to a naturally-occurring, synthetic, recombinant, or chemical change or difference to the primary, secondary, or tertiary structure of a polypeptide or polynucleotide, as compared to a reference polypeptide or polynucleotide, respectively (e.g., as compared to a wild-type polypeptide or polynucleotide). Polypeptides and polynucleotides having such mutations can be isolated or generated using methods well known in the art.

As used herein, an “eNOS polypeptide mutant”, or grammatical equivalents thereof (e.g., eNOS mutant, mutant eNOS, eNOS mutant polypeptide, mutant eNOS polypeptide), refers to an eNOS polypeptide or fragment, or variant thereof, having at least one variation or mutation in an amino acid residue corresponding to a position in a functional domain of a mammalian eNOS. In a preferred embodiment, the mutation is an amino acid substitution at a position corresponding to amino acid residue 495 of a human eNOS (SEQ ID NO: 1), where the amino acid substitution is preferably Ala or Val. In another preferred embodiment, the activity of the eNOS polypeptide mutant is increased or decreased, as compared to a reference eNOS polypeptide.

As used herein, a “functional domain” of an eNOS polypeptide is any amino acid residue, site, or region in the polypeptide associated with an eNOS activity, including but not limited to, e.g., a protein-binding domain (e.g., a calmodulin-binding domain, kinase-binding domain, or ligand-binding domain), phosphorylation site, myristolation side, reductase domain, or activation site.

As used herein, “eNOS activity” refers to any activity associated with the enzyme in cells including, but not limited to, e.g., NO production, calmodulin-binding, stimulating angiogenesis, ameliorating microvascular dysfunction, restoring vasomotor (vasodilator) activity of existing vessels, contributing to the remodeling/maturation of existing collaterals (arteriogenesis). An eNOS activity may also be any biological or cellular activity associated with the polypeptide, and more particularly, any such activity associated with a functional domain of an eNOS polypeptide. An eNOS activity may also be the modulation of an activity associated with the enzyme, including but not limited to, e.g., the modulation of any of the eNOS activities described above or known in the art.

As used herein, “modulation”, with reference to an eNOS activity, refers to an increase, decrease, induction, or repression of such activity. In some embodiments, such increase, decrease, induction, or repression of eNOS activity is relative to a reference molecule, e.g., eNOS wild-type or mutant polypeptide.

As used herein, “disease”, “condition”, or “disorder” refers to an undesirable condition in a cell, tissue, or organ of a patient where eNOS activity can be modulated to ameliorate the condition. Endothelial NOS is involved in a variety of physiological processes including, but not limited to, e.g., angiogenesis, vasodilation, immune regulation, inhibition of platelet aggregation, and relaxation of smooth muscle. Thus, modulating eNOS activity in a cell, tissue or organ of a patient in need of treatment can ameliorate a disease, condition, or disorder as described herein.

As used herein, a “patient” is a mammal and is preferably a human.

Polynucleotides Encoding eNOS Polypeptides

Endothelial nitric oxide synthase (eNOS, also known as ecNOS and NOS3) is one of three known isoforms of NO synthases. eNOS is found in, e.g., vascular endothelium, cardiac myocytes, blood platelets, various types of smooth muscle and cells of the immune system, such as e.g., T-cells, neutrophils, or macrophages. A polynucleotide encoding an eNOS polypeptide used in the methods of the invention can originate from any of the cells or tissues described herein, preferably from endothelium.

A polynucleotide encoding an eNOS used in the methods of the invention can originate or be derived from any mammalian source, e.g., human, mouse, guinea pig, dog, bovine, pig, rat or rabbit, preferably human. The present application is, in general, directed to the use of human eNOS (SEQ ID NO: 1) and to mutants thereof. However, one of skill in the art will recognize that the present disclosure also applies equally well to eNOS polynucleotides and polypeptides, and to mutants thereof, from other species, such as those mentioned above; and/or to the use of polynucleotides encoding an eNOS polypeptide to treat species other than humans.

Any form of polynucleotide encoding an eNOS polypeptide that can be introduced into a host cell (e.g., by transfection or transduction) and expressed therein is suitable for use in the methods of the invention.

Polynucleotides encoding an eNOS polypeptide of the invention include, e.g., a cDNA that encodes an eNOS polypeptide, or a DNA that codes without interruption for an eNOS polypeptide. A polynucleotide that “codes without interruption” refers to a polynucleotide having a continuous open reading frame (“ORF”) as compared to an ORF that is interrupted by introns or other noncoding sequences.

As used herein, the term “gene” means a segment of DNA involved in producing a polypeptide chain; it may include regions preceding and following the coding region (leader and trailer) as well as intervening sequences (introns) between individual coding segments (exons). The invention includes isolated genes (e.g., genomic clones) that encode eNOS polypeptides of the invention.

A polynucleotide of the present invention may be a recombinant polynucleotide, a natural polynucleotide, or a synthetic or semi-synthetic polynucleotide, or combinations thereof. The polynucleotides may be RNA, PNA, or DNA, e.g., cDNA, genomic DNA, and synthetic or semi-synthetic DNA, or combinations thereof. The DNA may be triplex, double-stranded or single-stranded. It can comprise hairpins or other secondary structures. The RNA includes mRNAs, polyadenylated RNA, total RNA, single strand or double strand RNA, or the like. DNA/RNA duplexes are also encompassed by the invention.

In one embodiment of the invention, the polynucleotide encoding an eNOS has the sequence of a naturally occurring polynucleotide, e.g., is a human polynucleotide. The sequence of polynucleotides encoding an eNOS is known for many species, e.g., human (Janssens et al. (1992) J. Biol. Chem. 267, 14,519-522) and bovine (U.S. Pat. No. 5,498,539). A naturally occurring polynucleotide of the invention may have, for example, a coding sequence that is an allelic variant of a wild-type polynucleotide sequence. As is known in the art, an allelic variant is an alternate form of a polynucleotide sequence which may have a substitution, deletion or addition of one or more nucleotides, which in general does not substantially alter the function of the encoded polypeptide. Polynucleotides comprising naturally occurring single nucleotide polymophisms (SNPs) may also be used in methods of the invention.

In another embodiment, a polynucleotide used in a method of the invention may be a variant of a naturally occurring eNOS polynucleotide. The term “variant,” as used herein, with regard to either a polypeptide or a polynucleotide, means that the variant retains substantially one or more of the activities or properties of a wild-type eNOS polypeptide or polynucleotide encoding it. That is, a variant is a polypeptide or polynucleotide that, when introduced into a cell, tissue or organ of a patient suffering from CLI by the methods of the invention can, to a measurable degree, ameliorate one or more of symptoms of CLI. A variant polynucleotide may encode either a wild-type or mutant eNOS polypeptide.

In another embodiment, a variant polynucleotide may encode a variant of a wild-type or mutant eNOS polypeptide, e.g., a polypeptide that comprises one or more deletions, insertions, additions, substitutions, inversions or truncations, or a combination thereof. Such a variant polypeptide may contain one or more amino acid substitutions, with a conserved or a non-conserved amino acid, preferably a conserved amino acid. Exemplary conservative substitutions, which preserve the general charge, hydrophobicity/hydrophilicity, side chain moiety and/or steric bulk of the amino acid substituted, include, e.g., Gly/Ala. Val/Ile/Leu, Asp/Glu, Lys/Arg, Asn/Gln, Thr/Ser and Phe/Trp/Tyr.

Variant polynucleotides encoding an eNOS used in the methods of the invention include, e.g., (i) one in which one or more of the nucleotides is substituted with another nucleotide, or which is otherwise mutated; or (ii) one in which one or more of the nucleotides is modified, e.g., includes a substituent group; or (iii) one in which the polynucleotide is fused with another compound, such as a compound to increase the half-life of the polynucleotide; or (iv) one in which additional nucleotides are covalently bound to the polynucleotide, such as sequences encoding a leader or secretory sequence or a sequence which is employed for purification of the polypeptide. The additional nucleotides may be from a heterologous source, or may be endogenous to the natural gene.

Polynucleotide variants belonging to type (i) above include, e.g., polymorphisms, including single nucleotide polymorphisms (SNPs), allelic variants, and mutants. Variant polynucleotides can comprise, e.g., one or more additions, insertions, deletions, substitutions, transitions, transversions, inversions, chromosomal translocations, variants resulting from alternative splicing events, or the like, or any combinations thereof.

Polynucleotide variants belonging to type (ii) above include, e.g., modifications such as the attachment of detectable markers (avidin, biotin, radioactive elements, fluorescent tags and dyes, energy transfer labels, energy-emitting labels, binding partners, etc.) or moieties which improve expression, uptake, cataloging, tagging, hybridization, detection, and/or stability.

Polynucleotide variants belonging to type (iii) above include, e.g., various lengths of polyA⁺ tail, 5′cap structures, and nucleotide analogs, e.g., inosine, thionucleotides, or the like.

Polynucleotide variants belonging to type (iv) above include, e.g., a variety of chimeric, hybrid or fusion polynucleotides. For example, a polynucleotide of the invention can comprise a coding sequence and, fused in the same reading frame, additional non-naturally occurring or heterologous coding sequence (e.g., sequences coding for leader, signal, secretory, targeting, enzymatic, fluorescent, antibiotic resistance, and other or diagnostic peptides); or a coding sequence and non-coding sequences, e.g., untranslated sequences at either a 5′ or 3′ end, or dispersed in the coding sequence, e.g., introns.

Polynucleotide variants used in methods of the present invention may also have a coding sequence fused in frame to a marker sequence that allows for identification and/or purification of the polypeptide of the present invention. The marker sequence may be, e.g., a hexa-histidine tag (e.g., as supplied by a pQE-9 vector) to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et a., Cell, 37:767 (1984)).

Other types of polynucleotide variants will be evident to one of skill in the art. For example, the nucleotides of a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability, etc. See, e.g., U.S. Pat. No. 5,378,825. Any desired nucleotide or nucleotide analog can be incorporated, e.g., 6-mercaptoguanine, 8-oxo-guanine, etc. Also, polynucleotides of the invention may have a coding sequence derived from another genetic locus of an organism, providing it has a substantial homology to a wild-type eNOS polypeptide or to one from another organism (e.g., an ortholog).

Other variant sequences, located in a coding sequence or in a regulatory sequence, may enhance the production of, or the function or activity of, an eNOS polypeptide of the invention.

Other variant polynucleotides have varying degrees of sequence homology (identity) to a wild-type polynucleotide encoding an eNOS, or encode polypeptide variants having varying degrees of sequence homology (identity) to a wild-type eNOS polypeptide, provided, of course, that the variant polynucleotides or polypeptides retain the ability to ameliorate one or more symptoms of CLI in the methods of the invention, when introduced into a patient suffering from that condition. That is, the polynucleotides or polypeptides are substantially homologous to the wild-type eNOS, or show substantial sequence homology (sequence identity) thereto. Thus, polynucleotides, polypeptides, and fragments thereof, within the present invention may contain polynucleotide or amino acid sequences which show at least about 65-70% sequence homology (identity) to wild-type polynucleotide or polypeptide, preferably about 70-75%, 75-80%, 80-85%, or 85-90% sequence homology (identity) thereto, and most preferably about 90-95% or 95-99% sequence homology (identity) thereto. The invention also encompasses polynucleotides and polypeptides having a lower degree of sequence identity, but having sufficient similarity so as to perform one or more of the functions or activities exhibited by the eNOS.

The term “substantially homologous,” when referring to polynucleotide sequences, means that the nucleotide sequences are at least about 90-95% or 95-99% or more identical.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100 [1-(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence.

The description herein for percent identity or percent homology is intended to apply equally to nucleotide or amino acid sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLASST) can be used. In one embodiment, parameters for sequence comparison can be set at score=100, wordlength-12, or can be varied (e.g., W=5 or W=20).

In a preferred embodiment, the percent identity between two amino acid sequences is determined using the Needleman et al. (1970) (J. Mol. Biol. 48:444-453) algorithm which has been incorporated into the GAP program in the GCG software package using either a BLOSUM 62 matrix or a PAM250 matrix, and a gap weight of 16, 14, 12, 10, 8, 6, or 4 and a length weight of 1, 2, 3, 4, 5 or 6. In yet another preferred embodiment, the percent identity between two nucleotide sequences is determined using the GAP program I the GCG software package (Devereux et al. (1984) Nucleic Acids Res. 12 (1):387) using a NWSgapdna CMP matrix and a gap weight of 40, 50, 60, 70, or 80 and a length weight of 1, 2, 3, 4, 5 or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al. (1994) Comput. Appl. Biosci. 10:3-5; and FASTA described in Pearson et al. (1988) PNAS 85:2444-8.

In accordance with the present invention, the term “substantially homologous,” when referring to a protein sequence, means that the amino acid sequences are at least about 90-95% or 97-99% or more identical. A substantially homologous amino acid sequence can be encoded by a nucleic acid sequence hybridizing to the nucleic acid sequence, or portion thereof, of a sequence encoding a mutant polypeptide of the invention, under conditions of high stringency.

Conditions of “high stringency,” as used herein, means, for example, incubating a blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity.

Other non-limiting examples of high stringency conditions include a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g.; overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

A polynucleotide fragment of the present invention may be of any size that is compatible with the invention, e.g., of any desired size that is effective to attain a therapeutic effect when introduced into a patient suffering from CLI. For example, a polynucleotide fragment of the invention may be slightly smaller than a full-length gene product. For example, a polypeptide of the invention may comprise at least about 10, 25, 50, 100, 200, 300, 400, 500, 600, 800, 1000 or 1200 amino acids. A polynucleotide of the invention may also be longer than a full-length cDNA, e.g., in the case of a fusion polynucleotide or a polynucleotide that is part of a genomic sequence.

Polynucleotides according to the present invention can be labeled according to any desired method. The polynucleotide can be labeled using radioactive tracers such as, e.g., ³²P, ³⁵S, ³H, or ¹⁴C. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

eNOS Polypeptides

The present invention further relates to eNOS polypeptides and mutants thereof.

Suitable mutations of a human eNOS (e.g. the human eNOS encoded by SEQ ID NO: 1), include, but are not limited to, e.g.,

(a) mutations in a phosphorylation site corresponding to amino acid residue 1177 where the residue is substituted to another amino acid, e.g., to Asp (S1177D) (see e.g., WO00/62605); and/or

(b) mutations in a myristolyation site corresponding to amino acid residue 2 where the residue is substituted to another amino acid, e.g, to Ala (G2A ) (see, e.g., Sessa et al. (1993), Circulation Research 72, 921-924. eNOS polypeptides mutated in the myristoylation site can be localized in the cytoplasm of a cell rather than at the membrane. Such mutants can be resistant (as compared to the wild-type protein) to pathological stimuli (e.g. oxLDL) which downregulate eNOS/NO production. Such properties can be an advantage, for use of the mutant polypeptide (or polynucleotide encoding it), in the treatment of CLI in where such external pathological stimuli exists;

(c) mutations in a calmodulin-binding site (e.g., amino acids 478-522 of SEQ ID NO: 1) corresponding to an amino acid residue in that domain, particularly, amino acid residue 495, which is known to be phosphorylated in mammalian cells. In preferred embodiments, the amino acid residue at 495 is substituted to Ala, Gly, Val, Leu, Ile, Pro, Phe, Tyr, Trp, Met, Ser, Cys, Glu, Gln, Lys, Arg or His, more preferably to Ala, Val, Leu, or Ile, and even more preferably to Ala or Val. Such mutants are discussed, e.g., in co-pending application U.S. Ser. No. 60/403,638, which is incorporated herein in its entirety.

Co-pending application U.S. Ser. No. 60/403,638 also discloses a variety of other mutations that, when present in eNOS, enhance its activity. Such mutants can be in the sites noted above, at other residues within those functional domains, or in other functional domains of the eNOS molecule.

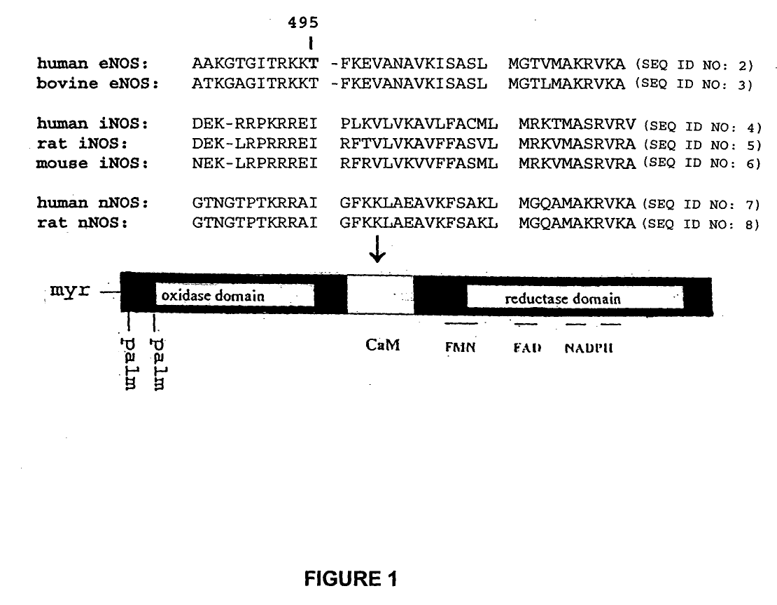

Functional domains of the eNOS polypeptide into which mutations can be introduced are well characterized (see FIG. 1) and include, e.g., proceeding from the N-terminus to the C-terminus, a consensus site for myristoylation; two sites for palmoylation; an oxidase domain; a calmodulin binding site (e.g., amino acids 494-517 of the human eNOS encoded by SEQ ID NO: 1), which comprises a consensus sequence for phosphorylation (of e.g., Thr-495 of the human eNOS encoded by SEQ ID NO:1); and a reductase domain which comprises a consensus sequence for phosphorylation (of e.g., Ser-1177 of the human eNOS encoded by SEQ ID NO: 1).

Further mutations, other than substitution of Thr-495, can be made in the calmodulin binding site, DPWKGSAAKGTGITRKKTVKEVANAVKISASLMGTVMAKRVKATI (e.g., SEQ ID NO: 2), amino acid residues 478-522 of SEQ ID NO: 1). The comparable sequence in other species can be slightly different, particularly in residues that are N-terminal to the phosphorylation site (see FIG. 1, SEQ ID NOS: 3-7). Each amino acid in this motif can be changed to any of the other 19 natural amino acids, or to a non-natural amino acid. In some embodiments, the mutation is not a conservative one, e.g., Gly/Ala, Val/Ile/Leu, Asp/Glu, Lys/Arg, Asp/Gln, Thr/Ser or Phe/Trp/Tyr. In one embodiment, one or more residues of an eNOS polypeptide of the present invention which are located immediately C-terminal of the amino acid residue corresponding to Thr-495 of SEQ ID NO: 1, e.g., residues 496 and 498, are replaced by Arg or Lys.

The invention also includes eNOS polypeptides in which one or more amino acids are modified in other functional domains of the eNOS polypeptide. For example, one or more mutations can be introduced into one or more of the catalytic domains (e.g., the oxidase or reductase domain) or regulatory regions (e.g., the autoinhibitory loop), or any of the functional domains described elsewhere herein or known in the art. The additional mutations can be any of the types of mutations described elsewhere herein.

Polypeptide mutants having a combination of two or more of any of the mutations described herein can also be used in methods of the invention. In a preferred embodiment, an eNOS polypeptide mutant of the present invention has mutations in at least two functional domains and exhibits higher amounts of eNOS activity than does the starting or reference polypeptide. If desired, after one such additional mutation has been made and characterized, the process can be repeated until a third mutation, in one of these two functional domains or a different functional domain, is made and characterized, and so forth. In some embodiments, the activity of an eNOS polypeptide mutant (e.g., stimulation of NO production) continues to increase with each additional mutation. In a preferred embodiment, a human eNOS mutant polypeptide comprises combinations of amino acid substitutions at positions corresponding to Thr-495 of SEQ ID NO: 1 (e.g., to Ala, Val, Leu or Ile, preferably Ala or Val) and Ser-1177 of SEQ ID NO: 1 (preferably to Asp); and an amino acid substitution at Gly-2 in the myristoylation site (preferably to Ala). In a most preferred embodiment, a polynucleotide of the present invention encodes an S1177RD mutation and G2A mutation; a S1177D mutation and T495V or T495A mutation; a G2A mutation and T495V or T495A mutation; or a S1177D mutation, G2A mutation and T495V or T495A mutation.

When the eNOS polypeptide of the present invention originates from a mammal other than human, comparable (equivalent) residues in the eNOS can be mutated. The comparable residues in such organisms are well-known. For example, the 495 residue of the human protein corresponds to residue 494 of the mouse polypeptide, residue 498 of the guinea pig polypeptide, residue 497 of the dog, bovine or pig polypeptide, residue 22 of the rat polypeptide, or residue 40 of the rabbit polypeptide.

Methods of generating such mutants are standard and well known in the art. These methods include, e.g., homologous recombination, site-directed mutagenesis, cassette mutagenesis, and PCR-based mutagenesis See, e.g., Sambrook et al., Molecular Cloning, CSH Press (1989) and Kunkel et al. (1985) PNAS 82, 488-492. The starting material for such mutations can be an eNOS cDNA from any animal, e.g., human, mouse, guinea pig, dog, bovine, porcine, rabbit, rat, ovine, equine, non-human primate, or other animal.

A variety of standard assays can be used to determine if mutants such as those described above exhibit increased eNOS activity. Assays of various eNOS activities can be performed; or one can determine directly the ability of a mutant eNOS polynucleotide to ameliorate one of more symptoms of CLI when using a gene therapy method of the invention. (see Examples)

eNOS converts L-Arg to NO, a gaseous second messenger molecule that is involved in, and/or serves a regulatory function in, many physiological responses. Without wishing to be bound to any particular mechanism, it is proposed that one or more of the activities mediated by eNOS contribute to the amelioration of symptoms of CLI when a polynucleotide encoding the eNOS is introduced into a diseased cell or tissue. For example, eNOS mediates, directly or indirectly (e.g., via NO produced by the enzyme): stimulation of angiogenesis; amelioration of microvascular dysfunction; stimulation of vasodilation; stimulation of collateral vessel development; enhancement of peripheral limb blood flow; inhibition of limb necrosis; inhibition of skin ulceration; enhancement of skin ulcer healing inhibition or prevention of platelet adhesion and aggregation (which can lead to, e.g., thrombus formation); stimulation of endothelial cell proliferation and migration; inhibition of leukocyte activation and adhesion or smooth muscle proliferation; modulation of an immune response; and scavenging of superoxide anion. Methods for assaying these and other eNOS activities are conventional and well known to those of skill in the art. See, e.g., methods for measuring the inhibition of limb necrosis, as evidenced by increased capillary density or vasomotor reactivity in the collateral vessel-dependent ischemic limb (see, e.g., Murohara et al. (1998) J. CGin. Invest., 101, 2567-2568. See also Examples herein.

Animal models for testing eNOS activity are standard and well-known in the art. For example, see Murohara et al. (1998 ibid); Couffinhal et al. (1998); Am J. Pathol 152, 1667-1669; Couffinhal et al., (1999) Circulation 99, 3188-3198; and Examples herein. Such assays include, e.g., mouse and rabbit models of surgical hindlimb ischemia, e.g., in which the surgery is performed in an eNOS deficient mouse. Methods to measure hindlimb blood flow and capillary density are also described in those references.

Recombinant Vectors Encoding eNOS Polypeptide Mutants

The present invention also relates to recombinant vectors containing polynucleotides of the invention, host cells containing recombinant vectors of the invention, and the production of polypeptides of the invention.

The invention relates to recombinant vectors into which a polynucleotide encoding an eNOS polypeptide is inserted. Such polynucleotides may be isolated directly from natural sources and cloned into appropriate expression vectors, or polynucleotides may be artificially engineered to have either naturally occurring or mutant polynucleotide sequences. Methods for mutating DNA, cloning and expressing it, or other molecular biological procedures referred to herein, are conventional and are described in any of a variety of textbooks and other reference materials. See, e.g., Sambrook et al. Molecular Cloning: A Laboratory Manual, Second Edition, Cold Spring Harbor, N.Y., (1989), Wu et al, Methods in Gene Biotechnology (CRC Press, New York, N.Y., 1997), Recombinant Gene Expression Protocols, in Methods in Molecular Biology, Vol. 62, (Tuan, ed., Humana Press, Totowa, N.J., 1997), and Current Protocols in Molecular Biology, (Ausabel et al, Eds.), John Wiley & Sons, NY (1994-1999).

The present invention relates to recombinant constructs that contain expression vectors (e.g., plasmid or viral expression vectors) into which a polynucleotide encoding an eNOS polypeptide as above has been inserted so as to be operatively linked to an appropriate expression control sequence(s) (e.g., promoters and/or enhancers, such that the encoded eNOS polypeptide is expressed.

Such constructs can be used in methods of either in vivo gene therapy or ex vivo (cell based) gene therapy, as described herein.

Appropriate expression control sequences, e.g., constitutive or regulatable promoters or other sequences (e.g., enhancers) known to control expression of genes in eukaryotic cells or their viruses, can be selected for in mammalian cells, such as human cells. A variety of such expression control sequences are known by those of skill in the art. Endogenous or heterologous expression control sequences can be used. The expression control sequences can be tissue-specific. In one embodiment, expression control sequences are derived from highly-expressed genes. Expression control sequences can be selected from any desired gene, e.g using CAT (chloramphenicol transferase) vectors or other vectors with selectable markers. Two appropriate vectors for such selection are pKK232-8 and pCM7.

Suitable promoters which can be used in the recombinant vectors of the present invention, include, e.g., the eukaryotic promoters: CMV immediate early, HSV thymidine kinase, early and late SV40, adenovirus promoters, LTRs from retrovirus, and mouse metallothionein-I. Selection of the appropriate vector and promoter is well within the level of ordinary skill in the art.

Transcription of the polynucleotide sequence encoding the eNOS polypeptides of the present invention can be increased by inserting an enhancer sequence into the expression vector. Enhancers are cis-acting DNA elements, usually about from 10 to 300 base pairs that act on a promoter to increase its transcription. Representative examples include the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors also include an origin of replication. An expression vector may contain a ribosome binding site for translation initiation, a transcription termination sequence, a polyadenylation site, splice donor and acceptor sites, and/or 5′ flanking or non-transcribed sequences. DNA sequences derived from the SV40 splice and polyadenylation sites may be used to provide required nontranscribed genetic elements. The vector may also include appropriate sequences for amplifying expression. In addition, expression vectors preferably contain one or more selectable marker genes to provide a phenotypic trait for selection of transformed host cells such as dihydrofolate reductase or neomycin resistance for eukaryotic cell culture.

Large numbers of suitable recombinant expression vectors are known to those of skill in the art, and many are commercially available. Suitable vectors include chromosomal, nonchromosomal and synthetic DNA sequences, e.g., derivatives of SV40; baculovirus; yeast plasmids; vectors derived from combinations of plasmids and viral DNA; and viral DNA such as vaccinia, adenovirus, adeno-associated virus, TMV, fowl pox virus, and pseudorabies. However, any other vector may be used as long as it is replicable and viable in a host. Appropriate cloning and expression vectors are described, e.g., by Sambrook et al., and other references discussed herein.

Recombinant vectors that are particularly useful for methods of gene therapy include recombinant retroviruses which are constructed to carry or express a selected polynucleotide of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651, EP 0 345 242 and WO 91/02805.

Packaging cell lines suitable for use with the above-described retroviral vector constructs may be readily prepared (see, e.g., PCT publications WO 95/30763 and WO 92/05266), and used to create producer cell lines (also termed vector cell lines) for the production of recombinant vector particles. Within preferred embodiments of the invention, packaging cell lines are made from human (such as HT1080 cells) or mink parent cell lines, thereby allowing production of recombinant retroviruses that can survive inactivation in human serum.

The present invention also employs alpha virus-based vectors. Such vectors can be constructed from a wide variety of alpha viruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250 ATCC VR-1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Suitable vectors can also be parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., PNAS 90:10613-10617 (1993).

In a preferred embodiment, adenoviral vectors are used. A variety of modified adenoviral vectors (e.g., of Ad5 or Ad2), particularly non-replicative vectors and/or helper independent viruses, are well-known in the art. Representative examples of adenoviral vectors include those described by Berkner, Biotechniques 6:616); Rosenfeld et al., Science 252:431-434 (1991); WO 93/19191; Kolls et al., PNAS 215-219 (1994); Kass-Eisler et al., PNAS 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5: 1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to (e.g., targeted) adenovirus, as described in Curiel, Hum. Gene Ther. 3:147-154 (1992), may be employed.

Appropriate DNA sequences may be inserted into a vector by any of a variety of procedures. In general, a DNA sequence can be inserted into an appropriate restriction endonuclease site(s) by standard procedures known in the art. Such procedures and others are deemed to be within the scope of those skilled in the art. Conventional procedures for this and other molecular biology techniques discussed herein are found in many readily available sources, e.g., Sambrook, et al., Molecular Cloning: A Laboratory Manual, Second Edition, Cold Spring Harbor, N.Y., (1989). See also Graham et al. (1988) Virology 63, 614-617 for a rescue recombination technique useful for the construction of, e.g., adenoviral gene delivery vehicles. The present invention also relates to host cells that are transformed, transfected, transduced with constructs (or infected with virus) such as those described above, and to progeny of said cells, especially where such cells result in a stable cell line that can be implanted (or re-implanted) into a patient in need of treatment of CLI.

Appropriate hosts for cell-based gene therapy include, but are not limited to: skeletal muscle cells, smooth muscle cells, bone marrow-derived stem cells, endothelial progenitor cells, fibroblasts, dendritic cells, umbilical cord blood derived cells, and endothelial cells. Introduction of a construct into a host cell in vitro can be effected by any suitable method, e.g., calcium phosphate transfection, DEAE-Dextran mediated transfection, lipofection, a gene gun, or electroporation (Davis, L., Dibner, M., Battey, I., Basic Methods in Molecular Biology, (1986)). Following transformation of a suitable host strain and growth of the host strain to an appropriate cell density, the selected promoter can be induced by appropriate means (e.g., temperature shift or chemical induction) if desired, and cells cultured for an additional period. The engineered host cells can be cultured in conventional nutrient media modified as appropriate for activating promoters (if desired), selecting transformants, amplifying the genes of the present invention and/or expanding the number of cells for use in cell-based gene therapy. The cell culture conditions, such as temperature, pH and the like, are those previously used with the host cell selected for expression, and will be apparent to the ordinarily skilled artisan.

CLI Gene Therapy Using eNOS Polypeptides and Polynucleotides

Critical limb ischemia (CLI) due to advanced peripheral arterial occlusive disease (PAOD) is characterized by, among other factors, progressive microcirculatory dysfunction and impairment of angiogenesis. The present invention relates to gene therapy methods of treating patients suffering from CLI (e.g., patients in Fontaine stage III or IV PAOD), using wild-type or mutant eNOS polypeptides or polynucleotides encoding such polypeptides to ameliorate one or more symptoms of CLI, including, but not limited to, e.g., microcirculatory dysfunction or impaired angiogenesis, activation of white blood cells, platelet aggregation, plugging of capillaries, endothelial dysfunction, reduced nitric oxide availability, endothelial damage, release of free radicals, tissue damage, necrosis, rest pain, increased ankle or toe systolic pressure and/or skin ulceration. A positive result of such therapy is improved angiogenesis and/or vasodilation of blood vessels and/or wound healing, in and around the damaged tissue.

Delivery of a polynucleotide or polypeptide of the present invention to a patient in need of 10 treatment can be accomplished in vivo (e.g. administering the polynucleotide or polypeptide directly to the patient) or ex vivo (e.g., introducing the polynucleotide or polypeptide to a cell, e.g., a cell taken from the patient to be treated or cell line derived from it, or a cell or cell line which is not from the patient to be treated, and then introducing the transfected cell to the patient.)

The methods of the invention involve administering an effective amount of a polynucleotide encoding an eNOS, in a gene delivery vehicle and/or in a transfected cell, to a patient in need thereof. By an “effective amount” is meant herein an amount that can achieve a measurable or detectable decrease or inhibition of one or more of the symptoms of CLI, e.g., the symptoms described elsewhere herein.

Methods of ex-vivo (cell-based) gene therapy are conventional. Appropriate cells containing expressible eNOS genes are described above. They can be introduced into a patient by any of a variety of suitable conventional methods, e.g., injection, grafting, intravenous administration, transplantation, implantation, delivery of cells encapsulated by a carrier, or various methods described herein for in vivo administration. Other delivery enhancing methods may be used. These included, but are not limited to: hyaluronidase injection, electroporation, and sonoporation. For in vivo administration, constructs as above, comprising an polynucleotide encoding an eNOS (sometimes called “gene delivery vehicles” herein), can be administered to patients in need thereof, using any of a variety of conventional procedures. The constructs can be delivered either locally or systemically. Suitable routes of in vivo delivery are well known to those of skill in the art and include, but are not limited to, intravascular, intramuscular, intraperitoneal, intradermal, intraarterial and oral methods. For typical methods, see, e.g., Jolly, Cancer Gene Therapy 1:51-64 (1994) Kimura, Human Gene Therapy 5:845-852 (1994); Connelly, Human Gene Therapy 1:185-193 (1995); and Kaplitt, Nature Genetics 6:148-153 (1994).

Gene delivery vehicles can be formulated into pharmaceutical compositions comprising conventional pharmaceutically acceptable excipients or carriers, using conventional methodologies. Formulations and excipients which enhance transfer (promote penetration) of an agent across cell membranes or which protect against degradation are also well-known in the art. For example, a delivery vehicle (for in vivo or ex vivo transfer, e.g., of a polynucleotide) may be delivered to a target cell by any of a variety of conventional procedures, including, e.g., liposome mediated transfection, e.g., in which the liposomes are cationic liposomes containing cholesterol derivatives such as SF-chol or DC-chol; formulated DNA (e.g., PINC); and transfection with lipofectamine, or the like. Typical methods are described, e.g., in U.S. Pat. No. 5,656,565; Mannino et al. (1988) BioTechniques 6, 682-690 and references therein; and Gao et at. (1991) Biochem Biophys Res Comm 179, 280-285.

In one embodiment, gene delivery vehicles which are administered to a patient suffering from CLI are administered locally to the site at which the disease condition is expressed. Such local delivery can avoid unwanted effects (e.g., side effects) resulting from, e.g., induction of NO in a non-disease related cell or tissue. For example, a polynucleotide of the invention may be delivered by a catheter inserted into the proximal portion of one or both femoral arteries, thereby effecting transfer into the cells of the skeletal muscles receiving blood flow from the femoral arteries. (See, e.g., U.S. Pat. No. 5,792,453). Direct injection into the peripheral vascular system, or into the diseased tissue, such as into skeletal muscle, can also be used, as can isolated tissue perfusion, e.g., isolated limb perfusion (ILP), wherein a closed circuit is created between the femoral artery and the femoral vein. (See, e.g., WO 01/03728).

In another embodiment, therapeutic polynucleotides are administered systemically, but are modified so that they are targeted to a cell, tissue or organ of interest, using conventional methods. For example, polynucleotides can be placed under the control of tissue-specific expression control elements, such as promoters or enhancer elements.

By fusing, for example, tissue-specific endothelial transcriptional control sequences to a transgene such as the eNOS genes of the invention within a construct, such as an adenoviral construct, transgene expression can be limited to endothelial cells. Examples of endothelial specific promoters include, e.g., the Tie-2 promoter (Schlaeger et al. (1997) Proc Natl Acad Sci 1; 94(7):3058-63), the endothelin promoter (Lee et al (1990) J. Biol. Chem. 265:10446-10450), and the eNOS promoter (Zhang et al. (1995) J Biol. Chem 270(25):15320-6). Flt-1 and Flk-1 promoters may be used as well (see e.g., Bu et a. (1997) J. Biol. Chem. 272:3216-32622). Other potential promoters include synthetic and natural skeletal muscle-specific promoters (see e.g., Hauser et al (2000) Mol. Therapy 2:16-24); Spc5-12 synthetic promoter (Li et al. Nature (1999) 17:241-245) and Patent WO 99/02737.

Other gene delivery vehicles and methods may be employed, including polycationic condensed DNA linked or unlinked to (e.g., targeted) adenovirus alone (for example, Curiel, Hum. Gene Ther. 3:147-154 (1992)); ligand-linked DNA (for example, see Wu, J. Biol. Chem. 264:16985-16987 (1989)); eukaryotic cell delivery vehicles cells (for example see U.S. Ser. No. 08/240,030, filed May 9, 1994, and U.S. Ser. No. 08/404,796); deposition of photopolymerized hydrogel materials; hand-held gene transfer particle gun (e.g. as described in U.S. Pat. No. 5,149,655); ionizing radiation (as described in U.S. Pat. No. 5,206,152 and in WO 92/11033); nucleic charge neutralization or fusion with cell membranes. Additional approaches are described in Philip, Mol. Cell Biol. 14:2411-2418 (1994) and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by beads. The method may be improved further by treatment of the beads to increase hydrophobicity and thereby facilitate disruption of the endosome and release of the DNA into the cytoplasm. Suitable liposomes that can act as gene delivery vehicles are described in e.g. U.S. Pat. No. 5,422,120, PCT Patent Publication Nos. WO 95/13796, WO 94/23697 and WO 91/14445, and EP No. 0 524 968. Further, non-viral delivery methods suitable for use include mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun (as described in e.g. U.S. Pat. No. 5,149,655); and the use of ionizing radiation for activating a transferred gene (as described in e.g. U.S. Pat. No. 5,206,152 and PCT Patent Publication No. WO 92/11033).

PINC (“protective interactive nonconsensing”) polymers such as poly(N-vinyl pyrrolidone) and poly(vinyl alcohol) have been designed to form complexes with plasmid DNA to (i) protect plasmids from rapid nuclease digestion, (ii) disperse and retain intact plasmid in target tissues such as skeletal muscle and (iii) facilitate the uptake of plasmid by muscle cells (Mumper et al., 1996, Rolland and Mumper, 1998). Mumper et al. (1998) showed that the PINC formulation enhances both the transfection efficiency to skeletal muscle (10 to 15 fold) and duration of expression (28 days) of plasmid DNA encoding a number of different transgenes including serum alkaline phosphatase (SEAP), human growth hormone, and human IGF-1 in rats. Abruzzese et al. (2000) have shown that the PINC formulation, in combination with electroporation, results in 100-fold increases in the production and secretion of recombinant proteins such as erythropoetin and vascular endothelial growth factor (FGG) from murine sketal muscle. Fewell et al. (2001) have demonstrated long term, sustained expression (out to 120 days) of human factor IX using a plasmid formulated with PINC following injection into the hind limb muscles of immune-deficient mice.

Another aspect is a method as above, further comprising administering, in addition to a polynucleotide encoding an eNOS polypeptide, one or more angiogenic regulators, either as polypeptides or as polynucleotides encoding those polypeptides, before, during or after administration of the eNOS. Many such angiogenic regulators are known to the skilled worker. They include, but are not limited to growth factors, transcription factors, vasoactive substances, chemoattractant molecules. Growth factors include e.g. HGF, VEGF (e.g., VEGF-2, VEGF-121, VEGF-145 or VEGF-165), FGF (e.g., FGF-1,-2,-4,-5), endothelial growth factor, epidermal growth factor, platelet-derived endothelial growth factor, TGF-α, TGF-β, PDGF, TNF-α or IGF, Developmentally regulated Endothelial cell Locus-1 (Del-1). Potential angiogenic regulators also, include, but are not limited to transcription factors (e.g. EPAS, HIF), vasoactive substances (e.g. kallikrein, C-type atrial natriuretic peptide (CNP), B1 receptor agonists) and chemoattractant factors (e.g. GM-CSF, MCP-1, IL-8) and other peptides (e.g., PR-39). Methods to prepare, administer, and test the effects of administration of such angiogenic factors, whether alone, or in combination with an eNOS polypeptide, are conventional. (See, e.g., Papapetropoulos et al., (1997) J Clin Invest 100, 3131-3139, Brock et a., (1991) Am J Pathol 138, 213-221, and Ku et al., (1993) Am J Physiol. 265, H 586-592, WO 01/03728, and references therein).

Such growth factors can be cloned into appropriate expression vectors (either into individual vectors or into a vector which also expresses an eNOS polynucleotide of the invention), using conventional procedures. (See, e.g., Rivard et aL, (1999) Am J Pathol 154, 355-363 for a method to induce angiogenesis by intramuscular gene therapy with VEGF). Cloned angiogenic factors can, of course, take the form of any of the functional variants or fragments discussed herein with reference to polynucleotides encoding an eNOS, except that the function that is retained is the angiogenic function of the wild-type angiogenic factors.

It can be advantageous to fuse, in phase and upstream of an angiogenic factor-encoding sequence, a sequence that aids in the secretion of the polypeptide from a host cell or that facilitates its attachment to a cellular membrane. Such suitable leader sequences are well-known to those of skill in the art.

In some embodiments, the method of gene delivery is by skeletal muscle injection.

Skeletal muscle injections of pro-angiogenic genes to stimulate new blood vessel formation in the ischemic leg is a convenient, relatively non-invasive approach for gene delivery. Skeletal muscle is composed of multinucleated, postmitotic myofibers, thus potentially facilitating an effective and long-term expression of transduced genes. Transient gene expression is sufficient for therapeutic angiogenesis, since the process of new blood vessel formation requires only 1-2 weeks. While it is possible that newly formed blood vessels may need more prolonged expression of growth factors for continued survival, in vivo data suggest that once blood flow is established through newly created capillaries/arteries, that these new vessels remain open without continued expression of the therapeutic transgene (1).

In some embodiments, the method of gene delivery is by adenovirus. Adenovirus has been used in angiogenic gene therapy experiments, because it can be easily produced in large quantities. The virus transfects dividing and non-dividing cells of various origins (organs and species) and it is capable of transient transgene expression. The transduction efficiency of adenovirus may depend on the presence of Coxsackie-adenovirus receptor (CAR) and _{αv} integrins. In addition, the muscle fascia and extracellular matrix of mature myofibers may act as a physical barrier that prevents effective viral gene delivery to muscle. Thus, permeabilization of fascia and extracellular matrix with digesting enzymes can enhance viral gene delivery. In addition, pretreatment with hyaluronidase can result in increased transgene expression using viral or plasmid delivery (U.S. Pat. Ser. No. 6,258,791). In addition, inflammation and ischemic damage and regeneration of myofibers occurring in skeletal muscle of patients and experimental animals with CLI may further facilitate viral gene transfer.

In another embodiment, plasmid vectors are used for gene delivery to skeletal muscle. Several approaches have been developed to enhance the efficiency of non-viral gene transfer to skeletal muscle including electroporation, plasmid polymer formulations such as PINC and PVP, and pre-treatments with compounds such as hyaluronidase or collagenase to increase access and penetration of the myofibers by naked DNA. Chemical formulation of plasmid DNA has greatly improved in vivo stability as well as uptake into target tissues. PINC (“protective interactive noncondensing”) polymers such as poly(N-vinyl pyrrolidone) and poly(vinyl alcohol) have been designed to form complex with plasmid DNA to (i) protect plasmids from rapid nuclease digestion, (ii) disperse and retain intact plasmid in target tissues such as skeletal muscle and (iii) facilitate the uptake of plasmid by muscle cells. Recently the PINC formulation has been shown to result in increases in the level and duration of expression of several transgenes in vivo. The ability to readminister the plasmid has also been demonstrated.

In some embodiments, local, catheter mediated VEGF-adenovirus gene therapy is used for gene delivery.

The following samples are offered by way of illustration and are not intended to limit the invention in any way.

### EXAMPLES

### Example 1

### eNOS Polypeptide Mutants and Recombinant Plasmid and Viral Vectors

Plasmid vectors encoding eNOS polypeptides having a single or double mutants were generated for plasmid vector delivery and expression of eNOS wild-type and polypeptide mutants in cells in vitro and in vivo. The mutants were generated using Kunkel site-directed mutagenesis directly in the eNOS polynucleotide sequence (Kunkel, T. A. PNAS 1985; 82:488-492). The mutations were confirmed by sequencing. The cDNAs of the wild-type mutant constructs were cloned into the plasmid vector, pShuttle-CMV, placing the polynucleotide encoding the eNOS polypeptide within a CMV expression cassette. Consequently, in these constructs the polynucleotide was operably linked to a CMV promoter such that the promoter drove the expression of the encoded eNOS polypeptide mutant in cells,

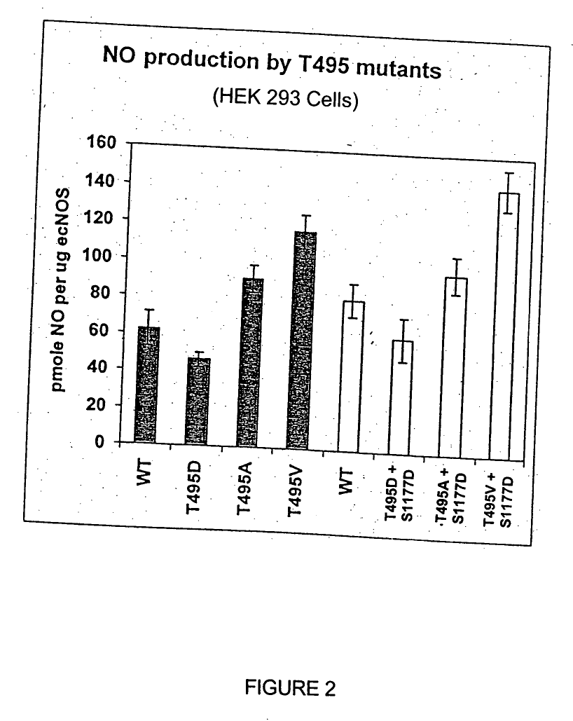

In the eNOS polypeptide mutants having a single amino acid substitution, the Thr corresponding to position 495 in the calmodulin binding site of human eNOS (see FIG. 1) was substituted to an Ala, Asp, or Val (designated mutants T495A, T495D, T495V, respectively). In the eNOS polypeptide mutants having a double amino acid substitution, the Ser corresponding to position 1177 was substituted to Asp and, additionally, the Thr corresponding to position 495 was substituted to Ala, Asp, or Val (designated T495A+S1177D, T495D+S1177D, T495V+S1177D, respec These mutations were confirmed by sequencing and tested for their ability to increase NO production in HEK 293 cells (Example 2 and FIG. 2)

Adenovirus vectors encoding eNOS polypeptides having the single and double mutations described above, were generated according to a method described by He et al (1998) PNAS 95(5), 2509-2514, and used for viral vector delivery of eNOS wild-type and polypeptide mutants in cells in vitro and in vivo. The pShuttle vectors carrying the polynucleotides encoding an eNOS polypeptide mutant (as described above) were co-transformed into E.coli. BJ5183, along with a plasmid containing an E1 and E3-deleted Ad5 genome. The adenovirus vector backbone was derived from Adenovirus 5. In this vector backbone, the E1 region of the Adenoviral sequence is deleted between nucleotide 454 and 3333, and a partial E3 deletion (nucleotides 30004 to 30750) is replaced with 645 bp foreign DNA. A polynucleotide can be inserted at the site of the E1-deletion such that the CMV promoter (at −632 to +7) and the SV40 polyadenylation signal are operably linked to the polynucleotide for expression of a polypeptide encoded by the polynucleotide.

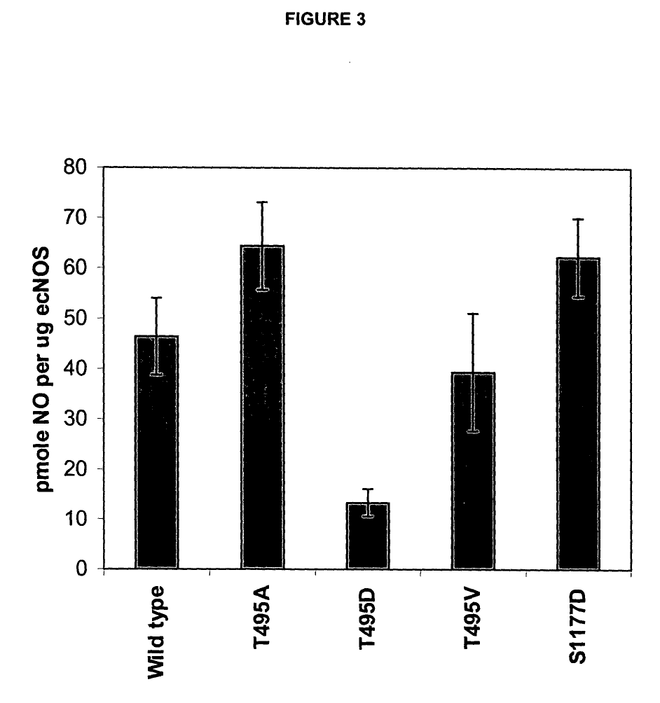

The resulting recombinant adenovirus plasmids encoding an eNOS polypeptide mutant were then selected and confirmed by restriction endonuclease analyses. The corresponding viruses were rescued by transfection of 293 cells with the recombinant adenovirus genomes excised from the plasmids and the viruses were then amplified in 293 cells, purified by standard CsCl gradient purification, and used for testing for NO production in HAEC (Example 3 and FIG. 3).

Additionally, the eNOS polypeptide mutant NOS 1177D (provided by Sessa et al., Yale University) has an amino acid substitution to Asp at a position corresponding to amino acid residue 1177 in the reductase domain of SEQ ID NO: 1. In order to test the activity of this eNOS polypeptide mutant in cells, a polynucleotide encoding this mutant was inserted into the adenovirus backbone (as described above in Example 1) at the position where the E1 position is deleted. The resulting recombinant vector, Ad5NOS1177D, encodes the eNOS polypeptide mutant NOS1177D. The recombinant vector Ad5NOS1177D was transfected into packaging cells and the resulting virus were plaque purified, and subjected to two rounds of amplification. Virus from the second amplification were used to inoculate a large-scale infection of HEK293 cells in a 3L-bioreactor. The resulting virus were then purified by two rounds of CsCl gradient separation and dialyzed against 10 mM Tris pH 8.0, 2 mM MgCl₂ and 4% sucrose. Aliquots of the purified recombinant virus were also used for testing NO production in HAEC (see Examples 3, 5, and 7).

Ad5EGFP is a control and is an adenovirus vector encoding the reporter gene, green fluorescent protein (GFP). It was prepared by Collateral Therapeutics, then amplified in HEK293 cells, and purified by FPLC. The purified virus was then dialyzed against PBS pH7.2 and 2% sucrose. Aliquots of the purified control virus were stored at −80° C. for use as a control in subsequent experiments (see Examples 5 and 7).

### Example 2

### Detection and Measurement of the Activity of eNOS Polypeptide Mutants in HEK 293 Cells

In order to test and measure the activity of eNOS polypeptide mutants in HEK 293 cells, plasmid vectors encoding an eNOS polypeptide mutant (described above in Example 1) were used to deliver and express the polypeptide mutants in HEK 293 cells. The HEK 293 cells were first plated in 6-well plates, in 2 ml per well of Growth Medium (Alpha MEM (Gibco 12561-056), containing 10% FBS (SeraCare), 2 mM additional L-glutamine and 50 μg/ml gentamicin. When the cells were about 75% confluent, they were transfected with a plasmid shuttle vector encoding the T495A, Thr495D, or T495V eNOS polypeptide mutant (as described above in Example 1), or a wild-type human (WT) eNOS (SEQ ID NO: 1), or both.

The transtection was performed by mixing 8 μg the plasmid shuttle vector encoding WT eNOS or a mutant eNOS, 60 μl Lipofectamine 2000 (Invitrogen) and 200 l OptiMEM (Gibco), and after incubating 30 minutes at room temperature, adding 111 μl of the mixture plus 420 μl OptiMEM to each well containing HEK 293 cells. After incubation at 37° C. for 2.5 hours, 2 ml of Growth Medium was added to each well.

After two days (cells incubated at 37° C., 5% CO₂), NO production by the cells was measured using chemiluminescence, after which the cells were lysed and the lysates assayed for eNOS protein content using an ELISA assay as described below. NO production was normalized to the amount of eNOS protein, in order to correct for variations in transfection efficiency between the different plasmids.

Measurement of NO Production.

The medium was removed, and each well was washed twice with 2 ml NO Analyzer Buffer (5 mM Na HEPES, 140 mM NaCl, 5 mM KCl, 1 mM MgCl₂, 10 mM glucose, 10 mM CaCl₂, 5 mM L-arginine, pH 7.5). Then the buffer was replaced with 1 ml of NO Analyzer Buffer containing 100 U/ml superoxide dismutase and 40 ng/ml VEGF. The wells were covered with parafilm, and after incubation for 30 minutes at 37° C., 0.8 ml of the buffer above the cells was injected into a Siemens NOA280 chemiluminesence detector for measurement of NO according to the manufacturer's instructions. Authentic NO gas was used as a standard. After NO measurements were completed, the remaining buffer on the cells was removed, and the cells were lysed in 0.6 ml Lysis Buffer (0.5% NP-40, 50 mM Tris-HCl pH 7.5, 1 μg/ml pepstatin A, 1 μg/ml leupeptin, 5 μg/ml aprotinin, 24 μg/ml Pefabloc SC (Boehringer Mannheim)) and stored at −20° C.

Measurement of eNOS Protein:

96-well ELISA plates (Costar 3590) were coated with 100 μl per well of Coating Antibody (rabbit polyclonal anti-eNOS), 5 μg/ml in 50 mM Na carbonate buffer, pH 9.5 and incubated overnight at 4° C. The polyclonal antibody (Babco) was collected from rabbits immunized with a peptide corresponding to residues 599 to 614 of human eNOS coupled to keyhole limpet hemocyanin, and purified using Protein G Sepharose (Amersham). Plates were blocked with 200 μl/well of 0.5% I-Block (Tropix) in PBS+0.01 % Tween 20 and incubated overnight at 4° C. Plates were then washed three times with 350 μl per well PBS+0.5 ml/L Tween 20. HEK 293 cell lysates containing eNOS were added to the plate, diluted five- or ten-fold into a final volume of 60 μl/well with Lysis Buffer, and incubated 1.5 to 2 hours at room temperature. Plates were then washed three times with 350 μl per well PBS+0.5 ml/L Tween 20.

The detection antibody, a monoclonal anti-eNOS antibody (Transduction Labs N30020) which was europium-labeled as described in Ref. 2, was added as follows: 125 ng/ml europium-labeled antibody in Wallac Assay Buffer (Wallac/PerkinElmer 1244-111), 100 μl/well. Plates were incubated 1.5 hours at room temperature. Plates were then washed three times with 350 μl per well PBS+0.5 ml/L Tween 20. Wallac Enhancement Solution (Wallac/PerkinElmer 1244-105) was then added, 100 μl/well. Plates were covered with plate sealers and stored overnight at 4° C., and then, after mixing for 10 minutes, plates were read in a Wallac 1420 VICTOR² multilabel counter (PerkinElmer Life Sciences), monitoring time-resolved fluorescence at 615 nm. (Aberle S. et al., Nitric Oxide 1, 226 (1997); Meurer J et al., Methods in Enzymology 359, 433-444 (2002).

The results indicate that the eNOS polypeptide mutants stimulated the production of NO in HEK 293 cells, and that the single mutants, T495A-and T495V, and double mutants, T495A+S1177D and T495A+S1177D, stimulated an increased level of NO production as compared to wild-type eNOS (FIG. 2).

### Example 3

### Detection and Measurement of the Activity of eNOS Polypeptide Mutants in HAE Cells

350,000 human aortic endothelial cells (HAEC) per well were plated in 6 well plates in 4 ml of EGM growth medium (Cambrex) containing 10% FBS. Cells were cultured at 37° C. in 5% CO₂. The next day adenovirus (2×10⁹ total viral particles per well, approximately 2×10⁷ infectious particles per well) encoding wild-type or mutant eNOS (Thr495Ala, Thr495Asp, Thr495Val or Ser1177Asp) was added to each well. After 4 hours of incubation with the virus, the medium was removed and replaced with 2 ml of EBM growth medium (Cambrex) supplemented with 0.1% gelatin and 30 μM sepiapterin (Sigma). After 20 hours, nitric oxide production by the cells was measured using chemiluminescence, after which the cells were lysed and the lysates assayed for eNOS protein content using ELISA as described below. Nitric oxide production was normalized to the amount of eNOS protein, in order to correct for variations in expression level as a result of differences in transfection efficiency with the different adenovirus constructs carrying the different eNOS mutants.

The results indicate that the eNOS polypeptide mutants stimulated the production of NO in HEK 293 cells, and that the single mutants, T495A and T495D stimulated an increased level of NO production as compared to wild-type eNOS (FIG. 3). The results of this study differs from those described in the Example 2, in that the cells were stimulated with VEGF in order to stimulate NO release, whereas as a calcium ionophore was used in the study described in Example 2. In addition, the adenovirus infection produced more eNOS protein per cell (and more nitric oxide) than transfection with plasmid DNA. Consequently, over expression of eNOS in this study may have contributed to the level of NO activity observed for the eNOS polypeptide mutants.

Human aortic endothelial cells contain endogenous wild-type eNOS, but the amount of NO produced from the over-expressed mutant eNOS is approximately 20 times that of the endogenous eNOS. In the data described in Examples 2 and 3, the nitric oxide production is normalized to the amount of eNOS protein, so the eNOS polypeptide mutants have activities in the same range. It is possible that the level of NO production detected between the different eNOS polypeptide mutants, using adenoviral vectors and plasmid vectors and different cell types, is due to other limiting factors in the cell, such as cofactor availability.

Thus, further testing of eNOS polypeptide mutants in HAEC can further elucidate the effects of eNOS expression and activity in different cell types.

### Example 4

### Generation of an eNOS-KO Mouse CLI Model

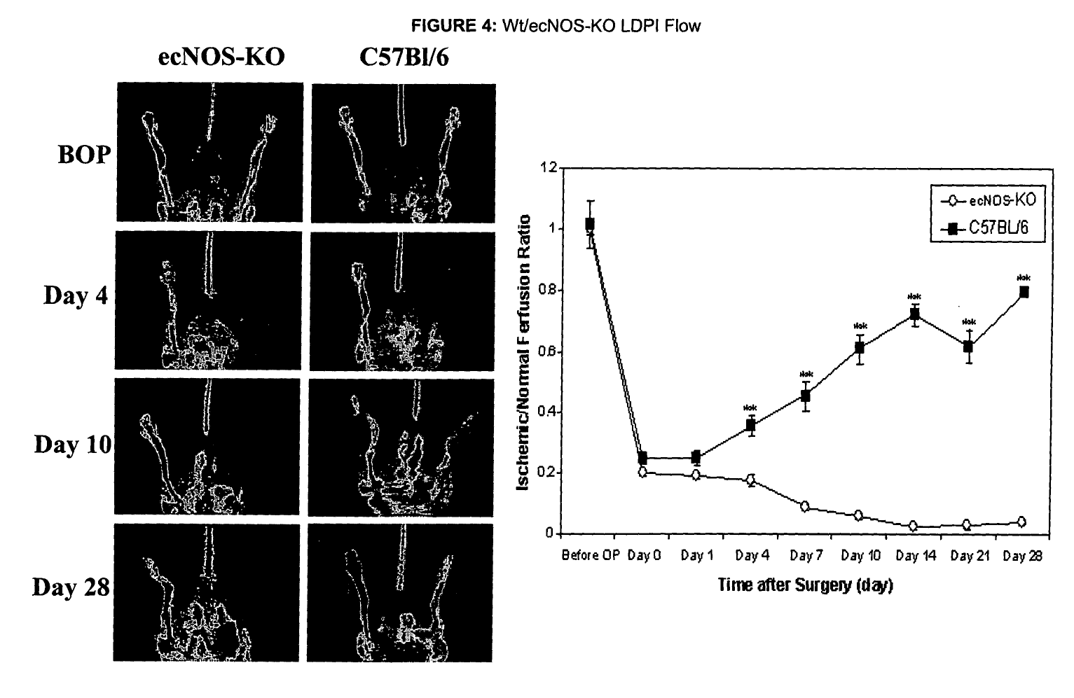

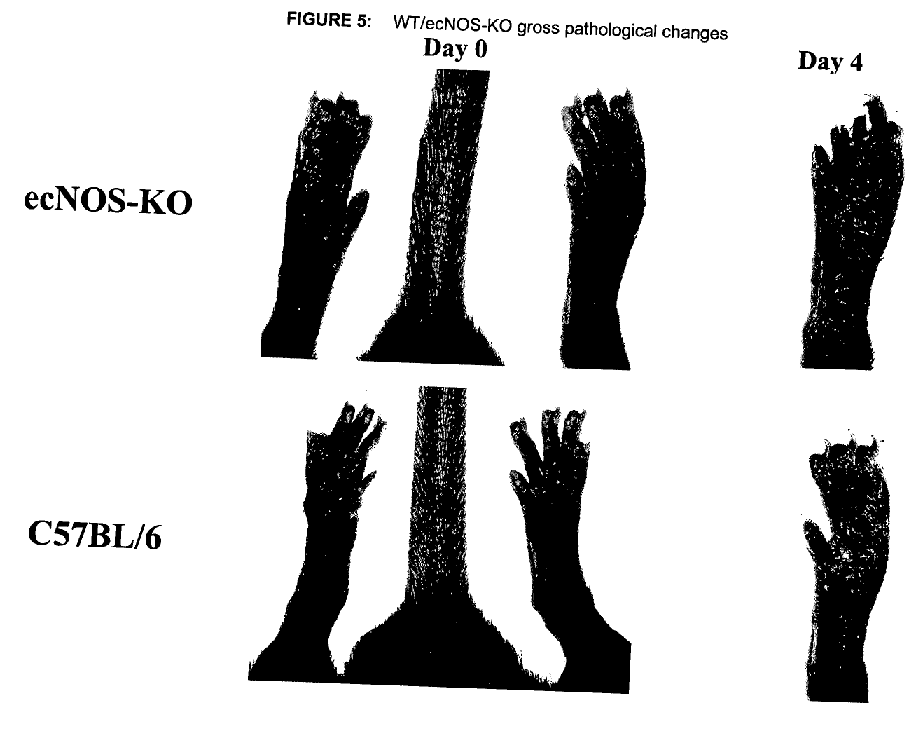

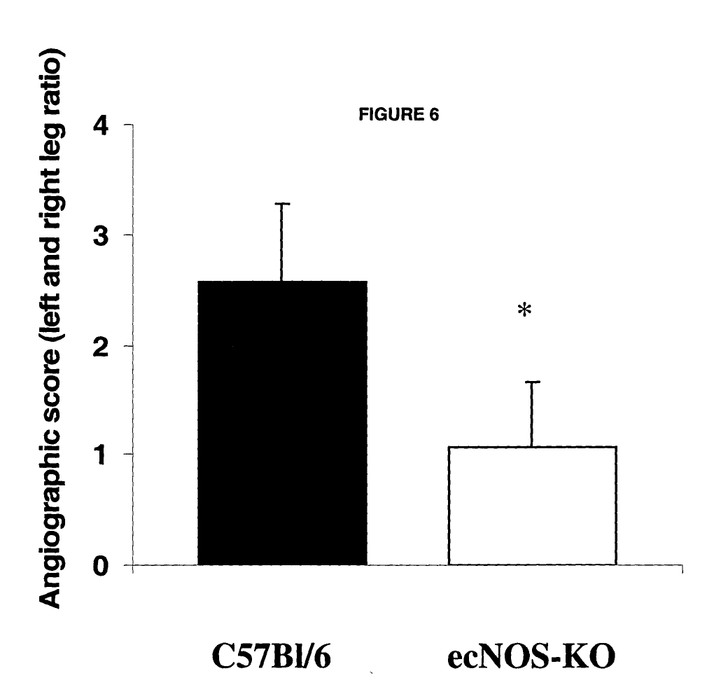

To develop an animal model of CLI, unilateral surgical resection of the femoral artery was performed in 3-12 months old wild-type (WT) and eNOS deficient (eNOS-KO) male mice (see Material and Method, below). Changes in superficial hind limb blood flow were measured using a laser Doppler perfusion imaging (LDPI) system before surgery, and 1, 4, 7, 10, 14, 21, and 28 days following surgery (FIG. 4). Quantitative angiography was performed on the ischemic limb 1, 5 and 10 days following surgery. In WT mice, blood flow was significantly impaired in the first week after occlusion, but was restored to 80% of pre-ischemic baseline levels at day 28. However, in eNOS-KO mice, recovery of hind limb blood flow was absent (FIG. 4) and associated with ischemic limb necrosis (FIG. 5). The number of large collateral arteries was assessed using a validated angiographic score index and was 2.6 fold higher in WT (C57B1/6) than in eNOS-KO mice on day 10 (FIG. 6). By day 28 following surgery, the eNOS-KO mice had lost their lower ischemic hind limbs due to autoamputation of the necrotic limb. In contrast, none of the wild-type mice showed signs of limb necrosis.

These results demonstrated the important role of endothelium-derived NO in angiogenesis and tissue perfusion in limb ischemia.

Refinement of CLI Model

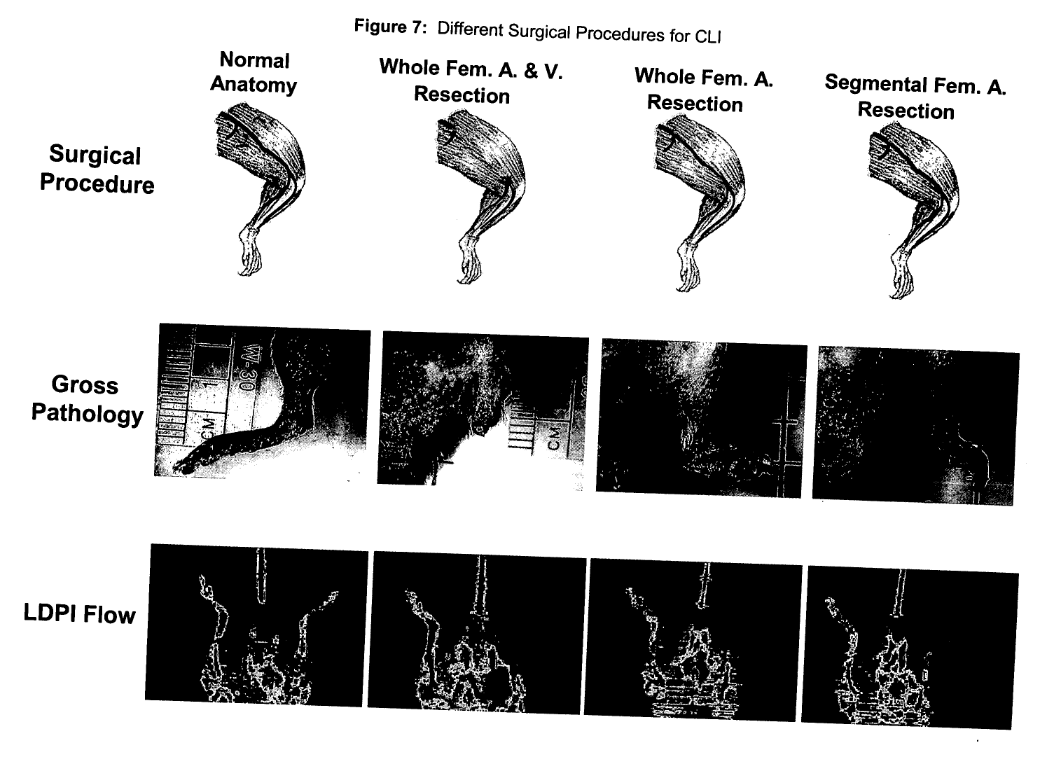

The severity of ischemic damage appeared to be dependent on the location and length of arterial resection and also on whether or not the femoral vein was left intact or removed. Resection of the entire femoral artery and vein resulted in severe ischemia and rapid loss of the entire limb in eNOS-KO mice. Removal of the femoral artery alone (femoral vein left intact) caused a more gradual ischemic necrosis. Segmental resection of the femoral artery alone resulted in less extensive ischemic necrosis (loss of toes or the distal limb) resembling human CLI (FIG. 7).

This model has been used to study subsequently the effects of interventions on blood flow and limb salvage.

Effect of Age on CLI

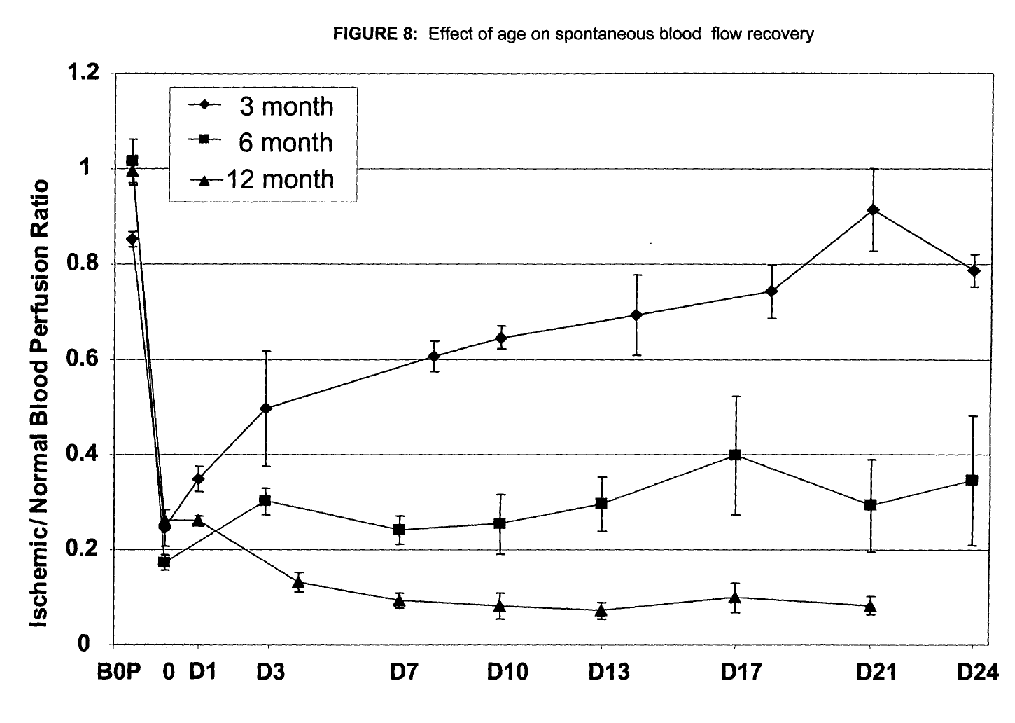

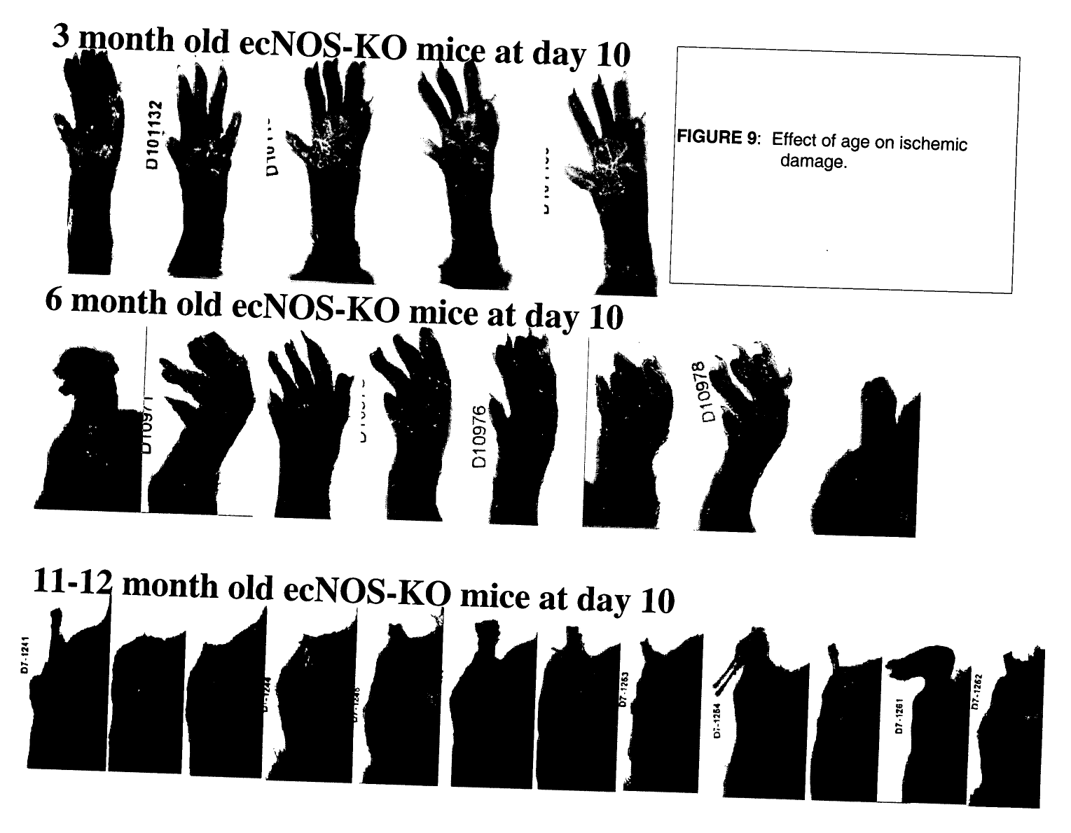

Using the CLI model described above, we observed that recovery of blood flow and the degree of ischemic injury were importantly dependent on the age of the animals. When performing segmental resection of the femoral artery, both the impairment in flow recovery and the extent of ischemic necrosis were much greater in older animals than younger eNOS-KO mice. eNOS-KO mice at 3 months of age behaved identically to WT controls. Full recovery of blood flow was observed in 14 days, without gross pathological changes. Animals at 6 month of age had significantly impaired blood flow recovery and trophic changes on the toes, resulting in loss of the foot in some cases (FIG. 8). At 11-12 months of age, the foot showed immediate discoloration after surgery and the mice developed rapidly progressing necrosis on the first day after surgery, which resulted in the loss of limb by day 10 in 90% of the animals (FIG. 9).

As a result of leg loss, some of the experimental endpoints, such as quantitative angiography or the LDPI flow, could not be evaluated in all experimental groups. In these instances efficacy of the treatment was determined by counting the remaining toes and legs of the animals at days 1, 4, 7, 10, 14, 21, and 28 following surgery.

Optimization of Gene Delivery

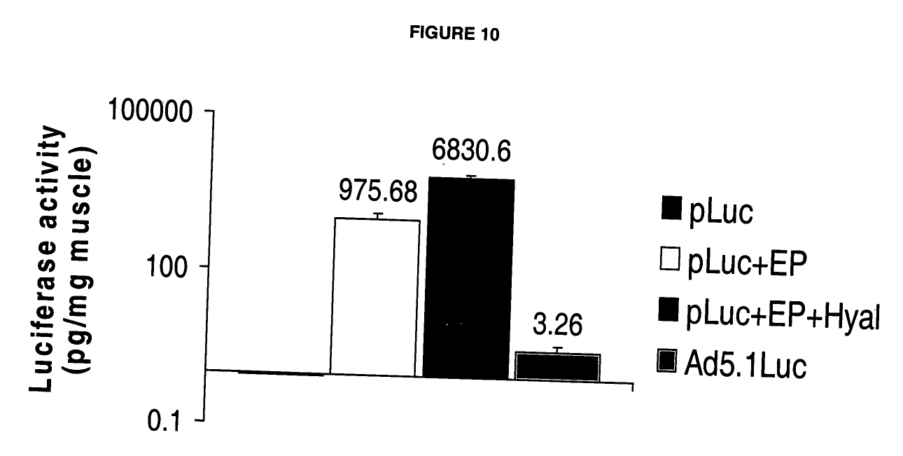

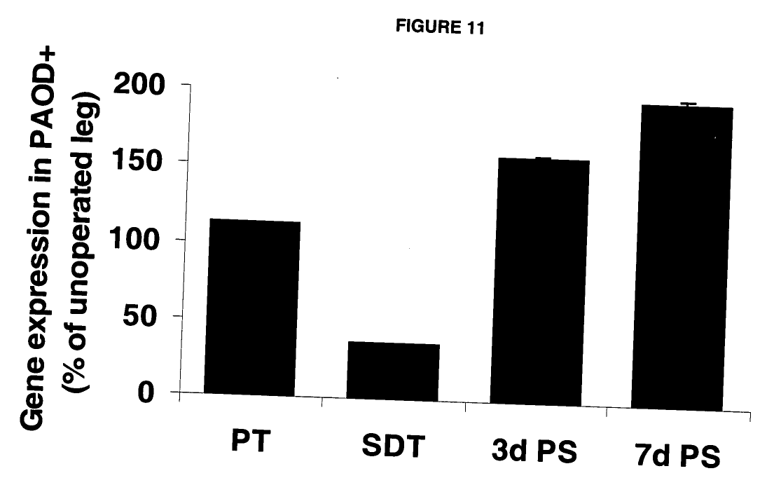

In some studies, plasmid DNA (pDNA) in combination with electroporation was used for gene delivery to cells. The studies were conducted in order to optimize: 1) DNA concentration, 2) volume, sites and number of injections, 3) electroporation conditions, 4) timing of treatment relative to surgery and 5) use of hyaluronidase pretreatment (FIGS. 10 and 11).

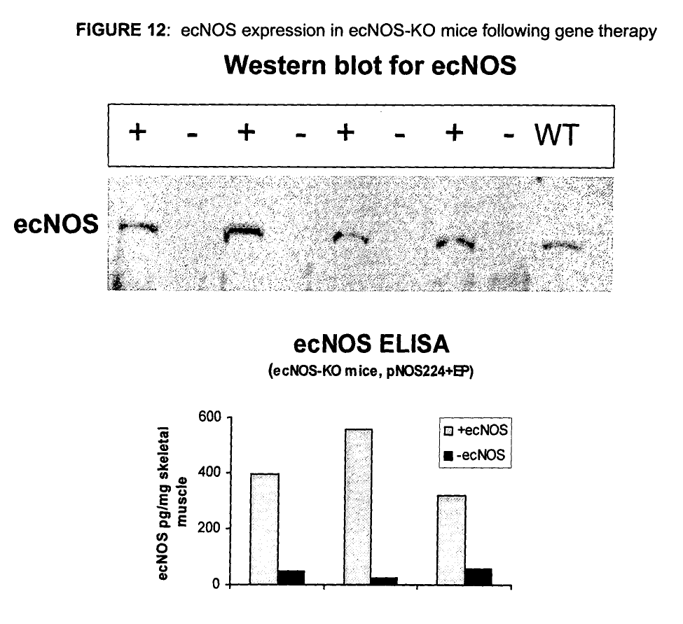

Gene delivery was optimized using pLuc, a plasmid containing the reporter gene luciferase, in WT mice and measured by determining luciferase activity in the adductor muscle homogenates at day 4 after injection. The best protocol was also tested using pNOS224 (a plasmid containing the NOS1177D gene) in eNOS-KO mice. In these experiments eNOS levels were determined by Western blot and an eNOS specific ELISA (FIG. 12).

### Example 5

### Efficacy of NOS1177D Treatment in eNOS-KO Mouse CLI Model

A. Prevention of Necrosis and Increase in Flow in 6 Month Old eNOS-KO Mice

This study was conducted using 6 month old male eNOS-KO mice. Mice were randomly distributed into two different treatment groups (n=8 in each group) after segmental femoral artery resection. One group was injected with the empty vector, the other with pNOS224 (containing the NOS1177D gene) in combination with electroporation on day 3 after surgery at two sites of the upper leg (adductor and quadricep muscles). LDPI flow measurements and pictures of the legs were taken at days 1, 4, 7, 10, 14, 21, and 28 following surgery. By the end of the study there was significantly higher rate of leg loss due to ischemic necrosis in the empty vector treated group (8/8) compared to the pNOS1177D treated animals (4/8) (p<0.05).

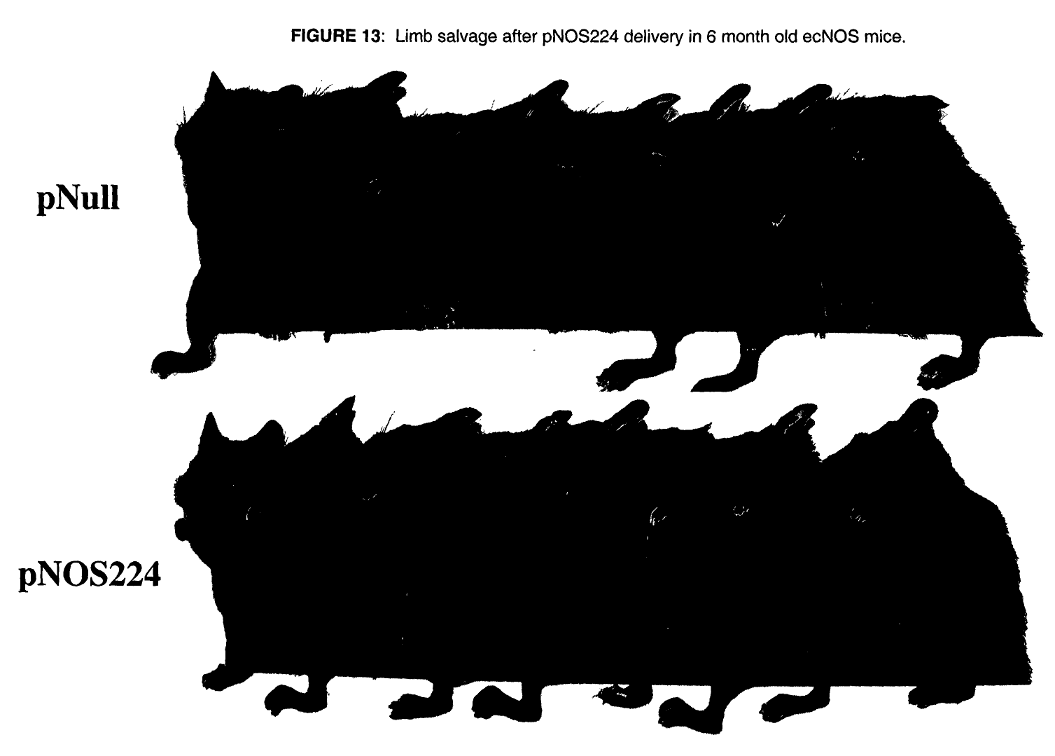

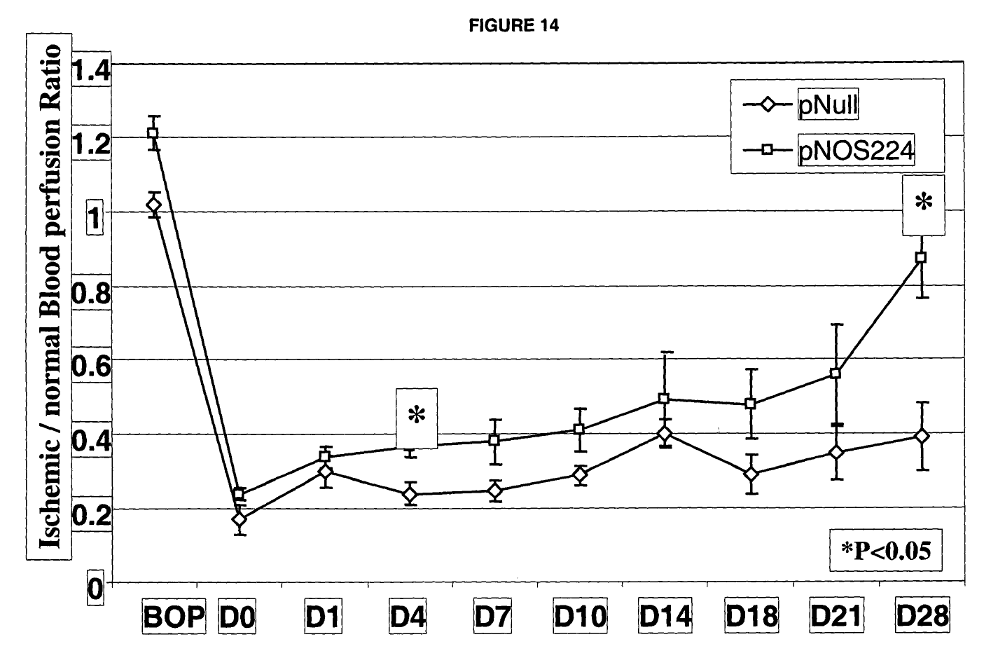

At day 28, animals were sacrificed and the limbs were collected for histomorphometric evaluation of treatment effects. By the end of the study there was significantly greater leg loss in the empty vector treated group (8/8) compared to the pNOS224 treated animals (4/8) (P<0.05) (FIG. 13). In addition, benefit was observed in ulcer healing in the pNOS treated animals (FIG. 14).

B. Limb Salvage in 11-12 Month Old eNOS-KO Mice

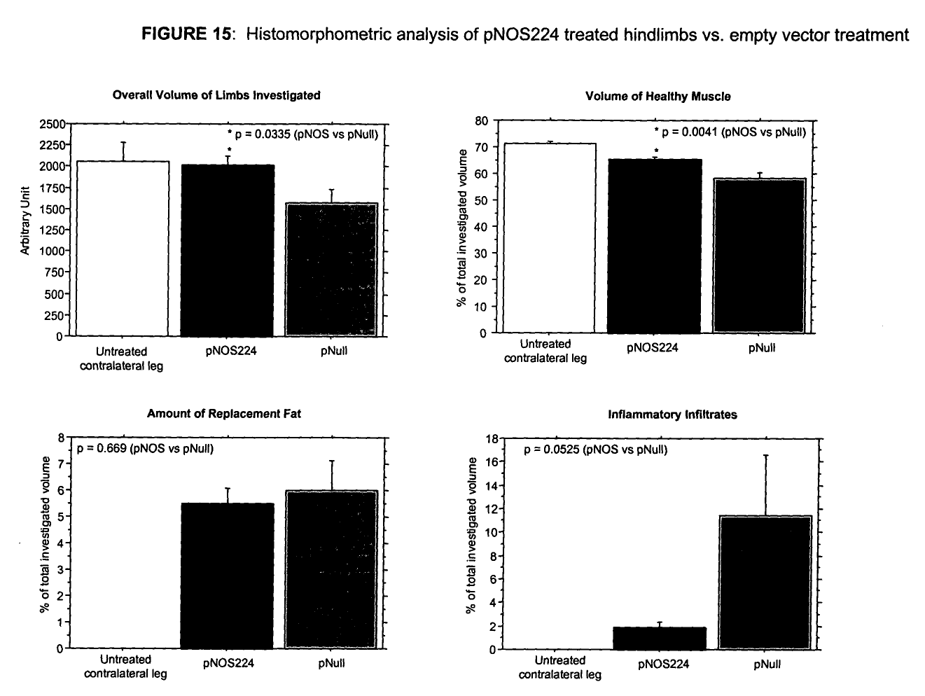

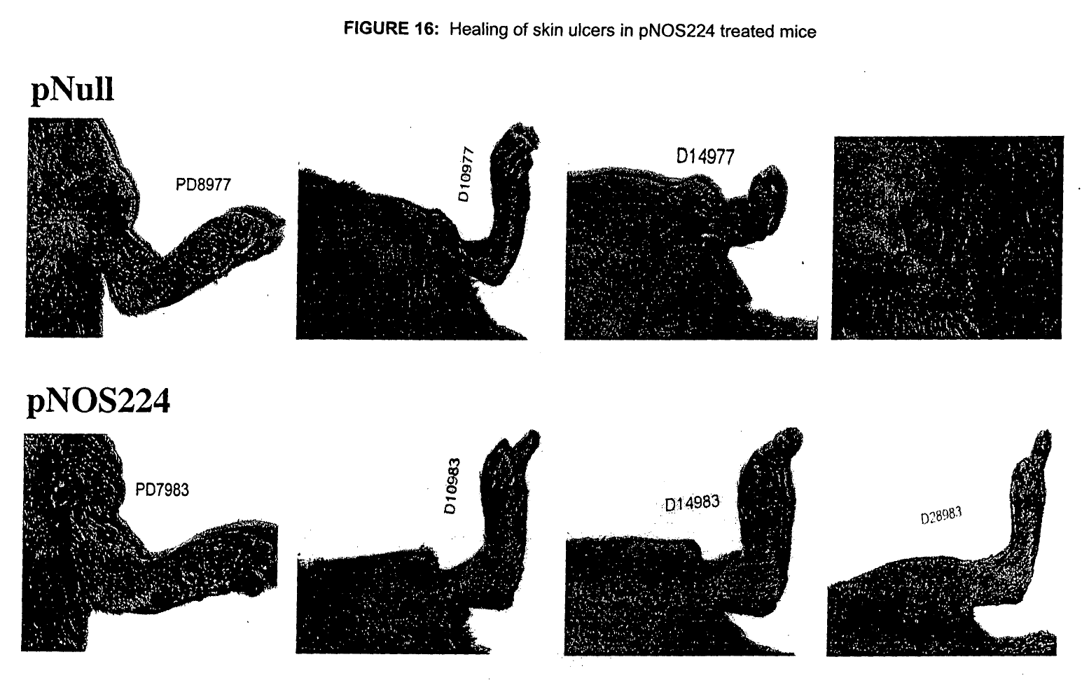

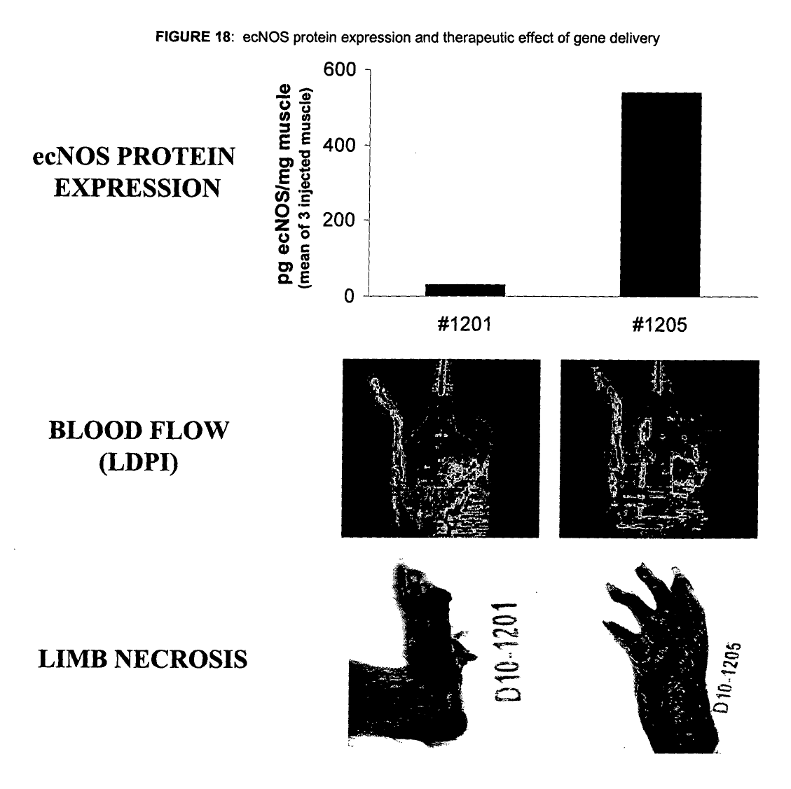

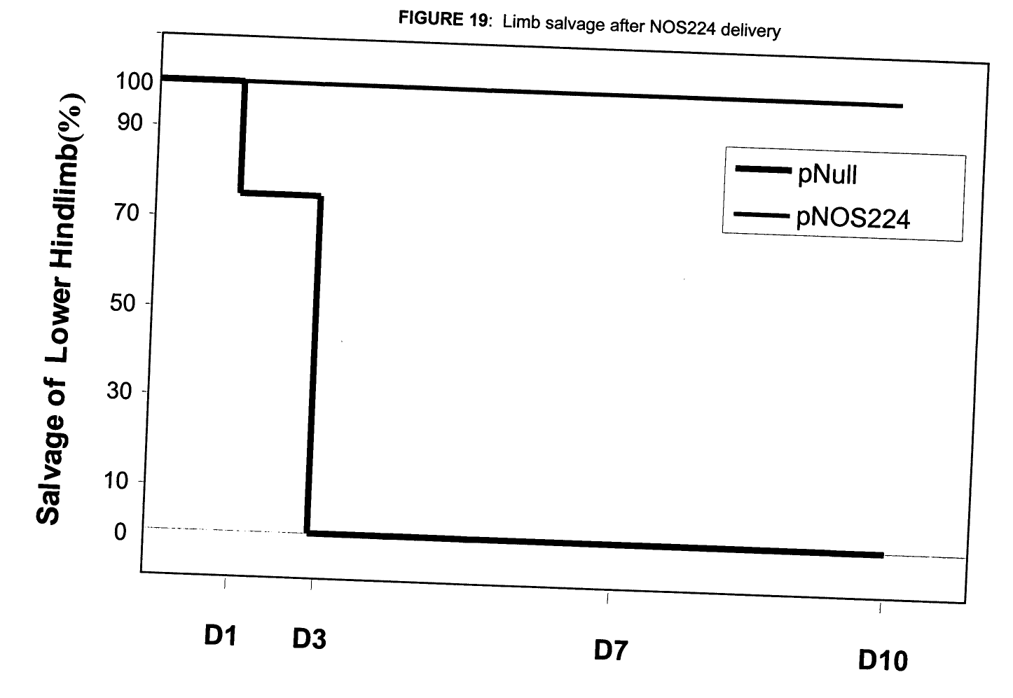

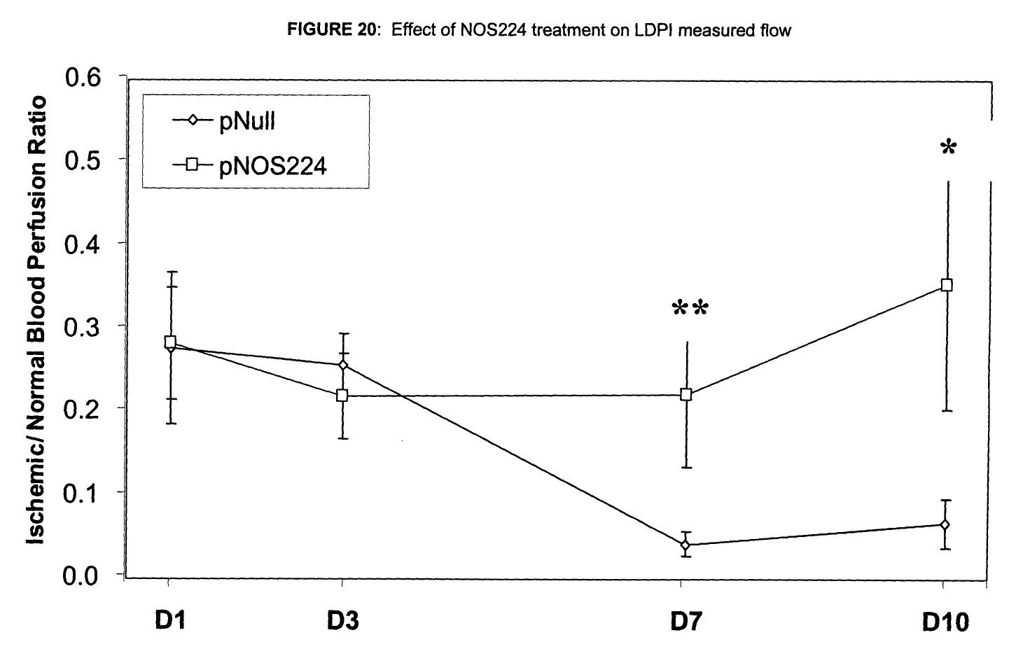

A second study was performed using 11-12 month old eNOS-KO mice. Due to the severity and rapid development of the ischemic damage in this age group, the gene delivery protocol was modified. The plasmid DNA was injected on the same day as the surgery at three sites (including the adductor, quadriceps, gastrocnemius muscles) and the muscles were pretreated with hyaluronidase at the sites of gene transfer 20 min prior to vector injection. In the empty vector treated group all animals lost their ischemic hindlimb due to severe necrosis and autoamputation by day 10 after surgery. In contrast, in the pNOS1177D treated group, there was a significant improvement in LDPI measured flow and the severity of ischemic necrosis was markedly reduced with salvage of all limbs (FIGS. 15, 16, and 18-21).

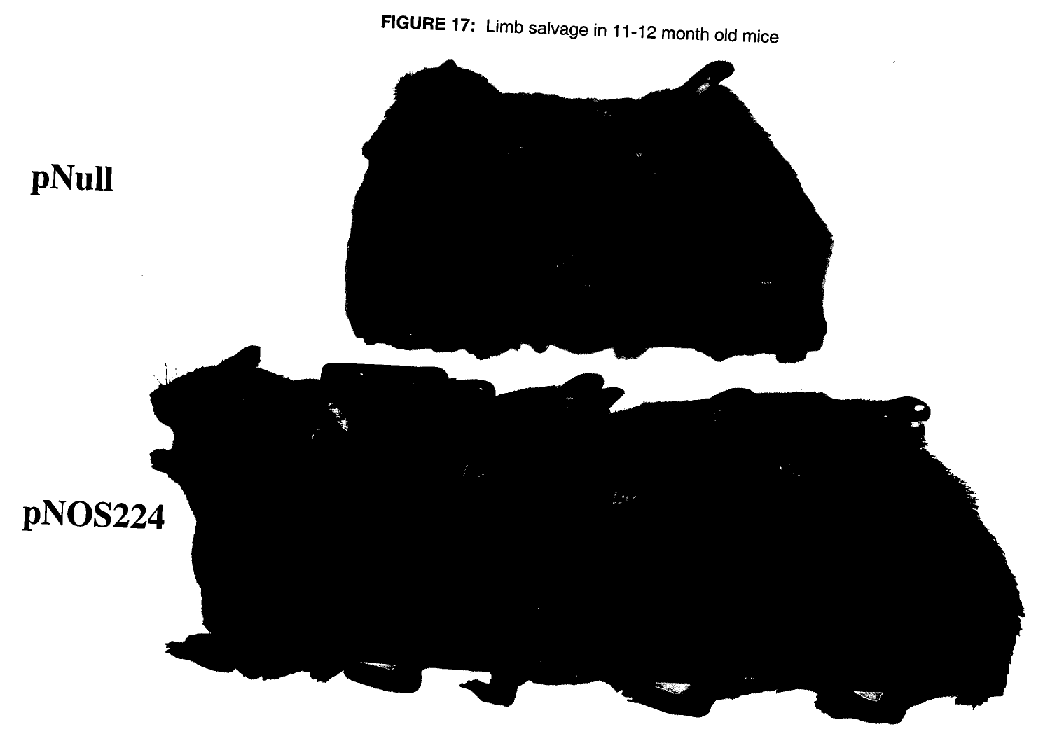

These changes in the protocol resulted in a significant improvement in gene expression (FIG. 17).

C. Conclusions

In conclusion, treatment with NOS1177D in eNOS-KO mice following surgical hindlimb ischemia augmented flow and attenuated/prevented ischemic necrosis resulting in limb salvage. These results prove the concept that, under disease conditions in which endogenous NO availability is severely impaired, NOS gene delivery has a therapeutic benefit.

### Example 6

### Development of New Critical Limb Ischemia Model in Rats Without Genetic Deficiency of eNOS and Testing the Efficacy of eNOS Gene Therapy Therein

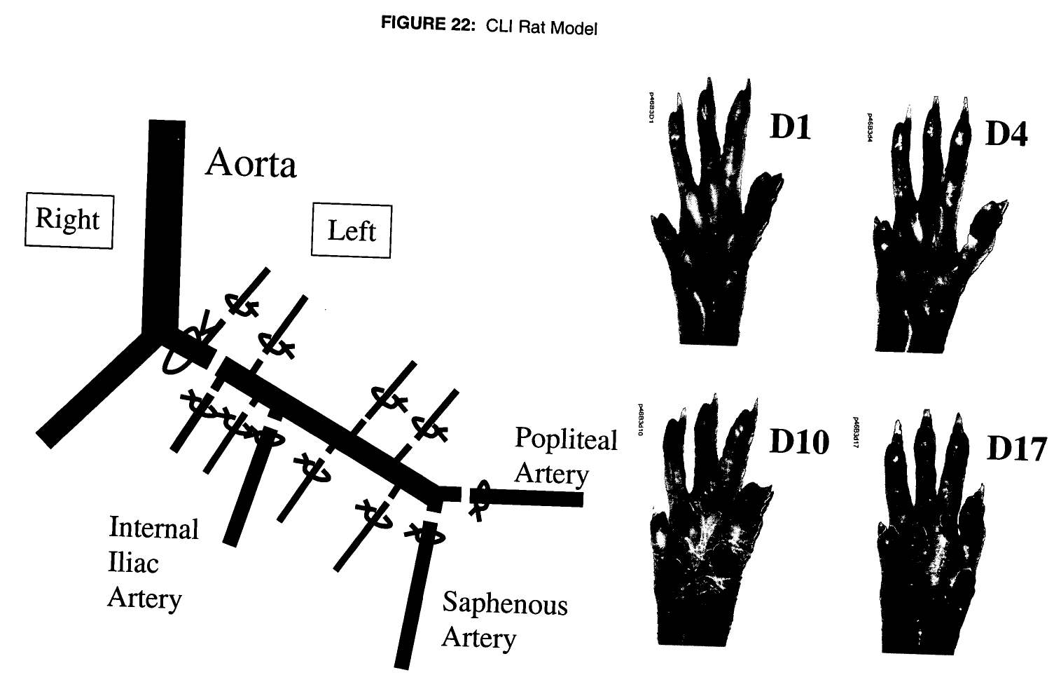

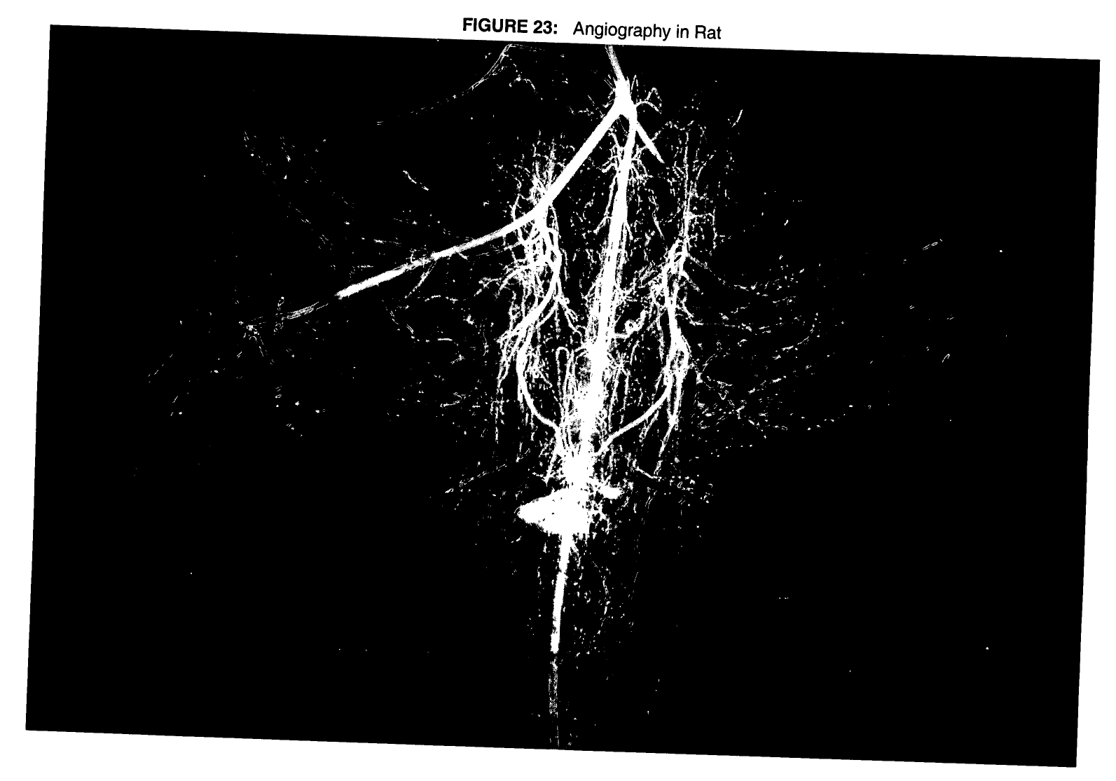

A novel model of CLI employing ligation and removal of the internal and external femoral artery and all side branches supplying the upper thigh area was developed in male adult Sprague-Dawley rats to evaluate the efficacy of NOS1177D in an animal model, which is not eNOS deficient (FIG. 22). FIGS. 22 and 23 illustrate gross pathological changes in the rat CLI model at day 1, 4, 10, 17, and 28 following operation.

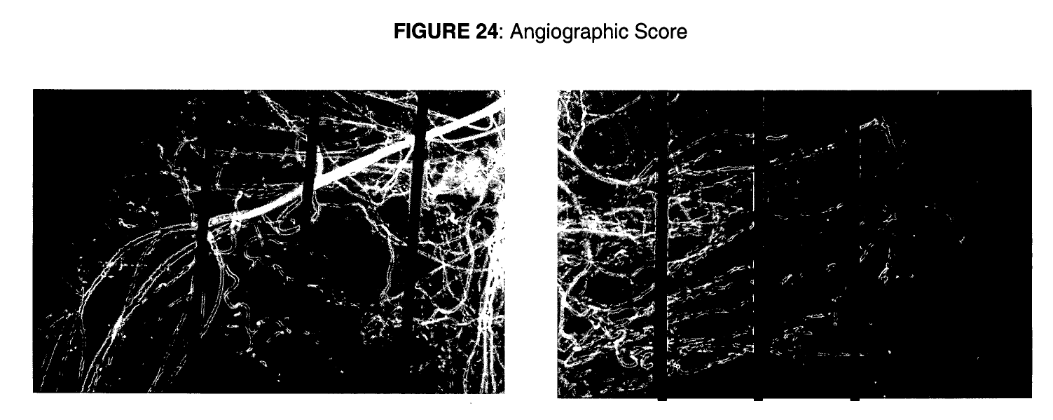

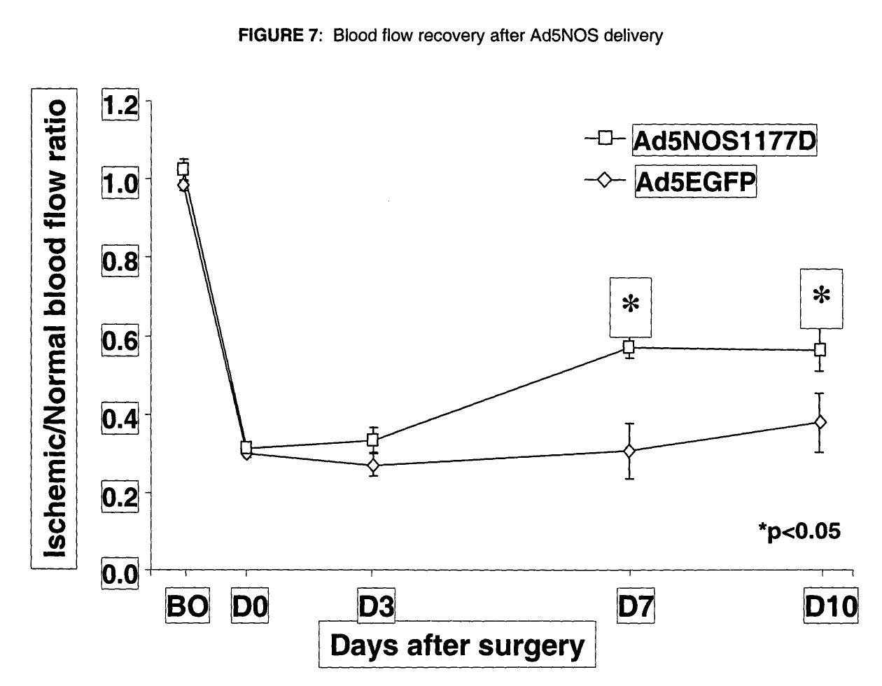

Further, a method of scoring arteriogenesis in the CLI rat model was developed. FIG. 24 illustrates angiography of a normal limb (left side of image) and an ischemic limb (right side of image) of the rat CLI model and FIG. 25 illustrates the angiographic scoring of arteriogenesis in a normal limb (left side of image) and an ischemic limb (right side of image) of the rat CLI model. To quantitate arteriogenesis, three straight lines started from internal ¼, middle, and external ¼ of the femur of the angiographic image were drawn at the medial thigh area of both the normal and ischemic limb, and the total number of arteries crossing these lines were calculated by two separate investigators blinded as to the treatment (e.g., plus or minus eNOS gene therapy). In order to minimize variations, the angiographic score was expressed as the ratio of the total artery number of left to right hind limb.

Surgical and eNOS Gene Therapy Protocol

Rats were distributed in two groups and immediately after surgery injected intramuscularly with 1×10¹¹ viral particles in 500 μl of PBS at three sites (adductor magnus, quadriceps—vastus medialis, and gastrocnemius), total 3×10¹¹ viral particles for three injections in each ischemic hind limb. Nine rats were injected with Ad5EGFP (as described in Example 1) as negative control and nine with Ad5NOS1177D (as described in Example 1) as treatment. Laser Doppler Perfusion Imaging (LDPI) was used to measure blood flow recovery on days 1, 4, 7, 10, 14 and 21 following gene transfer. Ischemic tissue damage was evaluated by taking photographs of the operated hind limb at the same time. Angiography was performed at the end of the treatment period.

In rats receiving NOS1177D, blood flow was restored from 31% of pre-surgery levels on day 1 up to 57% by day 10 after gene therapy. In the control animals, receiving the EGFP control gene, hind limb blood flow showed only minor recovery (30% on day 1 versus 37% by day 10), p=0.0226 at day 10 between the two groups (FIG. 25).

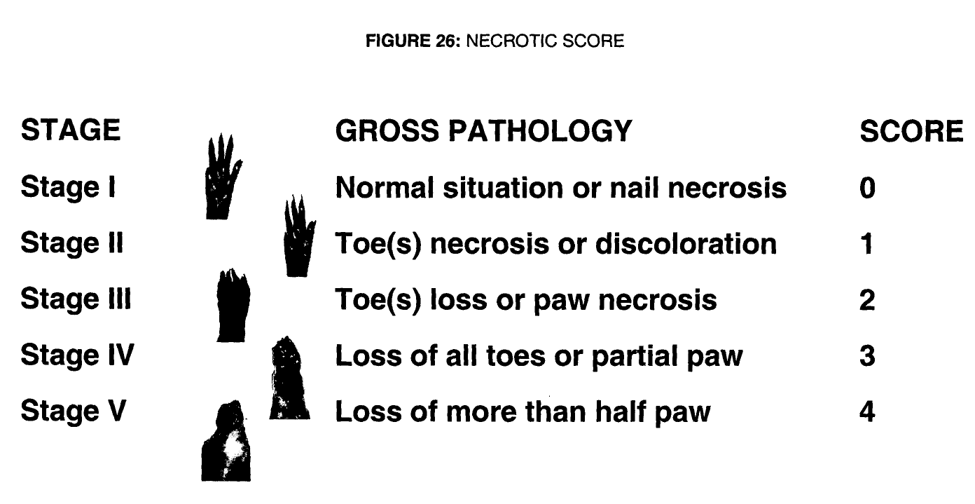

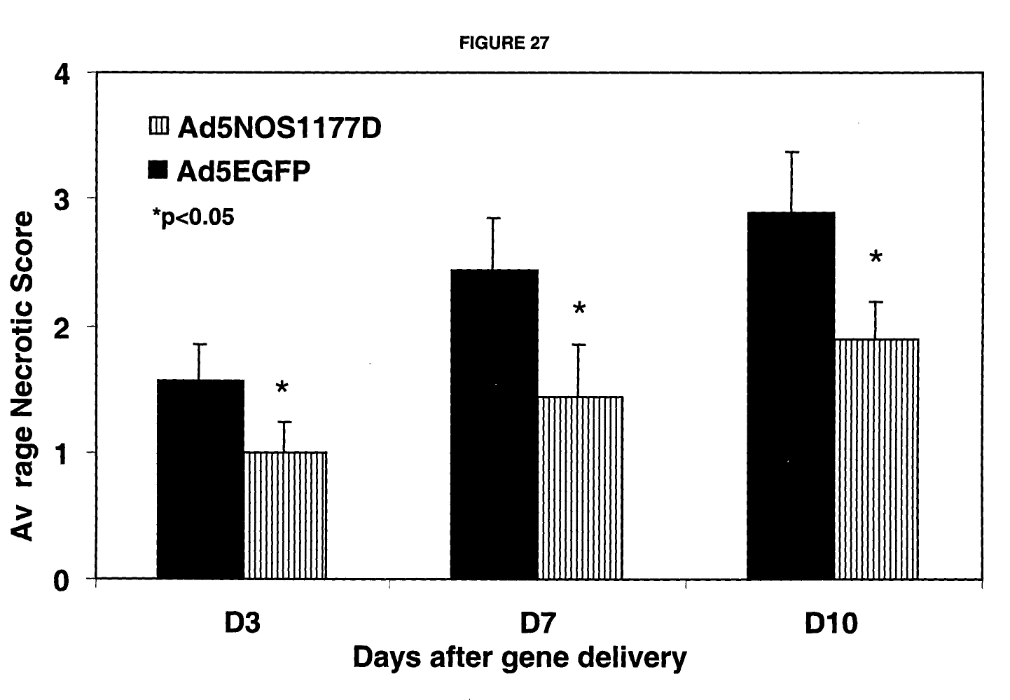

The necrotic score of the NOS treated group was based on gross pathological Stages I-V as illustrated in FIG. 26. The results indicate that NOS1177D gene delivery resulted in significant prevention of ischemic necrosis (score was 1.89 in NOS treated group versus 2.89 in control group at day 10, p=0.014) in association with a trend for increased eNOS expression (FIG. 27).

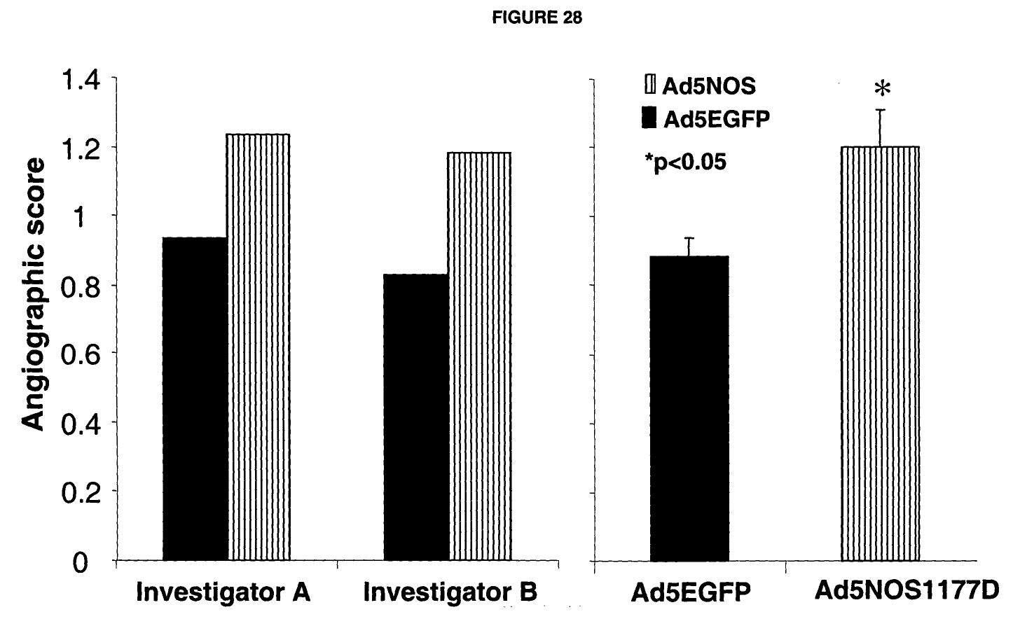

Further, arteriograms of the lower hind limb performed 14 days after gene transfer demonstrated more collateral vessels in the Ad5NOS1177D-treated rats compared to Ad5EGFP-treated rats (angiographic score was 1.21 versus 0.88, p=0.0147) (FIG. 28).

Thus, the results indicate that eNOS gene therapy in the rat CLI model could prevent ischemic necrosis and this effect correlated with increased expression of the eNOS polypeptide mutant in vivo, in the treated hind limb.

Material and Methods

Mouse Hindlimb Surgery

ENOS-KO male mice (Jackson Laboratory, Bar Harbor, Me.), 3-12 months of age, weighing 20 to 55 g, were used for all experiments. The animals were anesthetized with 1.5% isoflurane for the surgical procedure as well as for laser Doppler measurements of limb perfusion. Operative intervention was performed to create unilateral hindlimb ischemia in the mice. A skin incision (2 mm) was performed at the upper-portion of the left hindlimb overlying the femoral artery. The proximal portion of femoral artery was isolated, and all side branches ligated. The femoral artery and vein ligated and dissected to introduce the most severe ischemic damage. In other instances the femoral artery was isolated and dissected without the vein, or only a segment of the artery was excised (see FIG. “1”). The overlying skin was closed using surgical staples.

Laser Doppler Perfusion Image (LDPI) Analysis of Mouse Hindlimb Blood Flow

LDPI was used to evaluate perfusion of both the ischemic (left) and normal (right) hind limbs. Excess hair was removed from the hind limbs. Before initiating scanning, mice were placed on a heating plate at 37° C. to minimize variations in temperature. For each time point described, we used LDPI to perform consecutive measurements over the same region of leg. To minimize variables including ambient light and temperature, perfusion was expressed as the ratio of left to right hindlimb perfusion. Perfusion analysis was determined: before surgery; immediately after surgery; and on days 4, 7, 10, 14, 21, and 28 after surgery under anesthesia.

Mouse Skeletal Muscle Gene Delivery

Three days after surgery, mice were anesthetized with 1.5% isoflurane and injected intramuscularly with 80 μg of pNOS224 or empty plasmid vector in 50 ∥l of PBS into the adductor magnus and quadriceps muscles of the operated hindlimb, followed by electroporation using a caliper electrode (200 V/cm, 20 ms, 1 Hz, 8 pulses).

For the pretreatment with hyaluronidase, 20unit of the substance was injected in 50 μl of PBS into the adductor magnus, quadriceps, and gastrocemius and 2 hours later, each muscle was injected again with 80 μg of pNOS224 or empty plasmid vector in 50 μl of PBS at the same site, followed by electroporation using the caliper electrode (200 V/cm, 20 ms, 1 Hz, 8 pulses).

For adenovirus injection 1×10¹¹ vp was used in 50 μl injection volume into the adductor magnus muscle.

Mouse Skeletal Muscle Homogenization for Transfection Efficiency Studies

Muscle sections were excised, and stored at −80° C. until processed for Western, ELISA or enzyme activity analysis. Tissues were diced and homogenized using a Kinematic polytron homogenizer in lysis buffer containing 25 mM Tris pH 7.8, 10% glycerol, 0.2% NP40, Roche Protease Inhibitor mini-tablet (containing: 1 mM EDTA and inhibitors of serine and cysteine proteases). Cofactors for enzyme activity were also added: 4 μM each FAD, FMN, BH₄, 3 mM DTT, 3 mM CaCl₂, 0.125 μM Calmodulin. Most adductor muscles weighed 25 to 75 mg. After 5-10 minutes of centrifugation using an Eppendorf centrifuge at maximal speed, the supernatant was saved and the pellet homogenized again with 50 to 75 μl lysis buffer using Duall ground glass homogenizers. After a second centrifugation the two supernatants were combined. Third centrifugation resulted in a final concentration of 100 mg muscle per ml with final volume of 250 to 750 μl.

ENOS Specific ELISA

Concentration of eNOS protein was determined using a specific ELISA kit from R & D Systems (cat#DEN00). The capturing antibody was activated by the addition of 100 μl assay diluent. Aliquots of 50 to 100 μl of muscle homogenate or eNOS standard was added to each well. The plate was gently mixed for 2 hours at room temperature or stored at −4° C. overnight. Each well was washed three times using wash buffer from R&D Systems. Aliquots of 200 μl of horseradish peroxidase conjugated antibody was added for 2 hours. After thorough washing, aliquots of 200 μl color reagent was added. After 30 minutes the reaction was stopped with 0.5 N sulfuric acid and read at 450 nm. eNOS concentration was calculated using a standard curve from the eNOS standard provided.

NOS Activity Assays

Aliquots of 60 μl of each muscle homogenate or standard was combined with 20 μl of reaction mix containing NADPH, arginine and ¹⁴C-arginine for 1 hour at 37° C. Final concentration of NADPH was 0.2 mM, 5 μC of ¹⁴C-arginine is 20 μM. Reaction was stopped by filtering reactants through AG 50 W-resin suspended in 0.1 M sodium acetate pH 5.2 buffer containing 2 mM each EDTA and EGTA. The product, ¹⁴C-citrulline, was counted using Microscint-40 in a Wallac Micro Beta.

Western Blots of Mouse Skeletal Muscle Lysate for Determination of Different NOS Isoforms

SDS-PAGE:

For each sample, 30 μl of muscle homogenate was added to 10 μl 4× sample buffer (Invitrogen Cat. No. NP0007) containing 100 mM dithiothreitol. After heating for 7 to 8 minutes at 100° C., each sample was loaded onto a 10% Tris-glycine SDS-PAGE gel (BMA PAGEr Cat. No. 59102). Where needed, 1 μl of HEK cell lysate (in 40 μl of 1× sample buffer) containing recombinant human eNOS was added as a positive control. Prestained protein markers (10 μl of Invitrogen LC5725) were also loaded in one lane of the gel. Gel was run for 1.5 hours at 130V (constant voltage) in 1× Laemmli Running Buffer provided by the Berlex media prep department.

Blotting onto Nitrocellulose:

Proteins were transferred onto nitrocellulose for 2 hours at 20V (constant) using Novex/Invitrogen apparatus (apparatus was presoaked in transfer buffer). (Transfer buffer=100 ml 10× Transfer buffer, provided by Berlex media prep+200 ml methanol+700 ml H₂O.) After blotting, nitrocellulose was stored overnight at 4° C. in 20 ml TBS+5% nonfat dry milk. (TBS=0.02 M Tris-HCl, 0.12 M NaCl, pH 7.5).

Detection of Proteins Using Antibodies (All Steps at Room Temperature):

Blots were incubated in 1^{st} antibody (anti-eNOS or anti-bNOS mouse monoclonal BD/Transduction Labs diluted 1:2000 in TBS+0.1% Tween 20+5% nonfat dry milk) for 1 hr 15 min. Blots were then washed as follows: one 10-min and two 5-min washes in TBS+0.1% Tween 20. Blots were then incubated in 2^{nd} antibody (peroxidase-conjugated goat anti-mouse IgG, Chemicon Intl.AP308P or Roche 1814168, diluted 1:3000 in TBS+0.1% Tween 20+5% nonfat dry milk) for 1 hr. After washing as described above, plus an additional 5-min wash in TBS (no Tween), blots were incubated for 1 min in ECL reagent (Amersham Pharmacia RPN2106). Then blots were covered with Saran Wrap and exposed against Amersham Pharmacia Hyperfilm ECL (RPN 1674A) for 1 to 5 minutes and the film was developed.

Endothelial NO Release (NO-Analyzer)

350,000 HAEC/well were plated on 6 well plates in 4 ml of Clonetics growth medium (EGM) containing 10% FBS. The next day adenovirus (2×10⁹ total viral particles per well, approximately 2×10⁷ infectious particles per well) encoding wild-type or mutant eNOS (at Gly2 and/or Ser1177 positions) was added to each well. After 4 hours of incubation with the virus, the medium was removed and replaced with 2 ml of Clonetics basal medium (EBM) supplemented with 0.1% gelatin and 30 μM sepiapterin. The next morning, the medium was replaced with 2 ml fresh EBM-gelatin-sepiapterin with or without 100 μg/ml oxidized LDL (Intracel RP-047). After 6 hours, the medium was removed, and each well was washed twice with 2 ml NO Analyzer Buffer (5 mM Na HEPES, 140 mM NaCl, 5 mM KCl, 1 mM MgCl₂, 10 mM glucose, 10 mM CaCl₂, 5 mM L-arginine, pH 7.5). Then the buffer was replaced with 1 ml of NO Analyzer Buffer containing 100 U/ml superoxide dismutase and 40 ng/ml VEGF. The wells were covered with parafilm, and after incubation for 30 minutes at 37° C., 0.8 ml of the buffer above the cells was injected into a Siemens NOA280 chemiluminesence detector for measurement of NO. After nitric oxide measurements were completed, the remaining buffer on the cells was removed, and the cells were lysed in 0.3 ml eNOS ELISA Lysis Buffer. After storage overnight at −20° C., the lysates (5 to 20 μl of each) were analyzed for eNOS protein using the eNOS ELISA. Nitric oxide production was normalized to the amount of eNOS protein, in order to correct for variations in expression level between the different mutant adenoviruses.

Plasmids Constructs

1. pGL3-Control. Expression of the firefly luciferase gene in this plasmid is driven by the SV40 promoter/enhancer (Promega). The plasmid backbone is the pGL3-Basic backbone.

2. pcDNA3.1/eNOS224. Expression of the eNOS224 gene is driven by the CMV promoter of pcDNA3.1 and encodes an eNOS polypeptide mutant S1177D.

3. pcDNA3.1/hygro—is the control plasmid for pcDNA3.1 d (Promega).

Morphometric Analysis

ENOS-KO mice that had undergone ligation of the left femoral artery to mimic PAOD were sacrificed 28 days after surgery. The hind quarters were stripped of skin, fixed, placed in decalcifying solution for 2 days, and then blocked in a systematic random manner for morphometric evaluation of treatment effects. In brief, the legs were disarticulated at the hip joint and sectioned at 4 mm intervals. All sections were placed in a single cassette, dehydrated, and embedded with the lateral face of each section at the cutting face of the block. Five-micron-thick sections were cut and stained with hematoxylin and eosin (H&E) for morphometric evaluation using the C.A.S.T. stereology system.

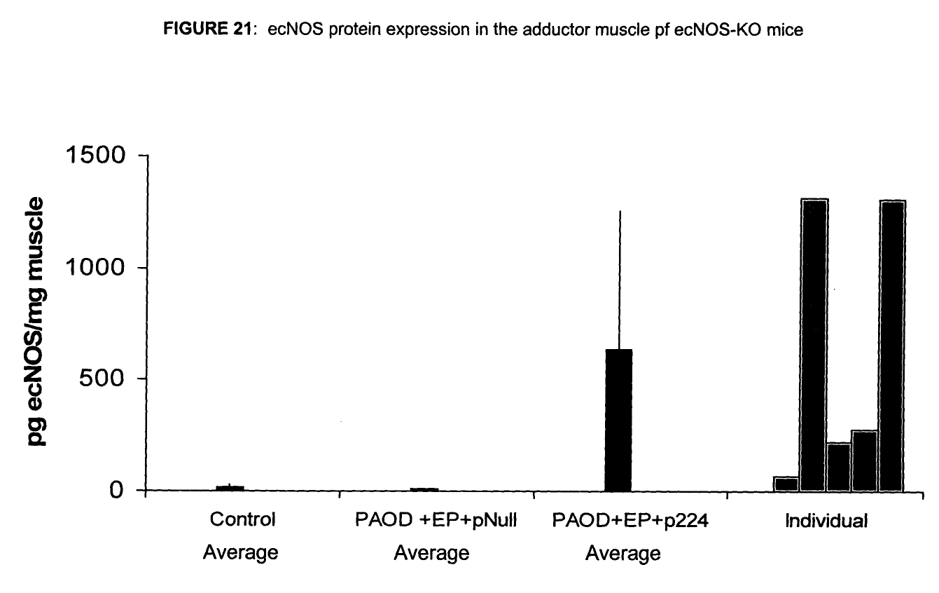

Both treated and untreated legs showed similar types of pathologic changes. The most consistent change was loss of muscle fibers with replacement of muscle volume by fat cells. This change was consistently present in the large muscle groups and was usually not associated with any evidence of active muscle degeneration. In some cases, however, active tissue destruction was still ongoing as evidenced by large numbers of acute and chronic inflammatory cell infiltrates around the replacement adipocytes as well as around degenerating muscle fibers. Areas of normal-appearing muscle, fatty replacement tissue, and inflammatory cell infiltrates were easily identified under the microscope. The area on each slide occupied by these tissues was individually quantified. All other regions on the slide (such as bone, skin, connective tissue, etc.) were grouped together in a category labeled “Other.” The volume of each of these regions was calculated as a percentage of the total volume of each leg. The results of these stereologic studies on both groups were compared by ANOVA (FIG. 21).

There were statistically significant changes in the following parameters: The overall volume of the eNOS-treated limbs was significantly larger than the null vector-treated, but not different from unoperated (control) limbs. The volume % of healthy muscle was also significantly decreased in null vector compared to eNOS vector. Correspondingly, the amount of replacement fat was not different in the null vector treated limbs as compared to the eNOS-treated limbs. The amount of inflammation was also increased in the null group, but this did not quite reach significance (P=0.0525).

These changes suggest that muscle fibers were lost immediately following femoral artery ligation and that the lost fibers were replaced by adipose tissue. In some areas, however, even the fatty tissue could not be supported by available blood flow, so tissue necrosis continued as evidenced by active inflammatory cell infiltrates. eNOS treatment ameliorated these pathologic changes.

1. A method of treating critical limb ischemia (CLI) comprising administering to a patient in need of treatment an effective amount of a polynucleotide encoding a mammalian eNOS polypeptide.

2. The method according to claim 1, wherein said eNOS polypeptide is a human eNOS polypeptide.

3. The method according to claim 2, wherein the amino acid sequence of said human eNOS polypeptide is SEQ ID NO: 1.

4. The method according to claim 3, wherein said eNOS polypeptide comprises at least one mutation at a position corresponding to an amino acid residue in said human eNOS that is phosphorylated in mammalian cells.

5. The method according to claim 4, wherein said eNOS polypeptide comprises a mutation at a position corresponding to amino acid residue 495 of SEQ ID NO: 1.

6. The method according to claim 4, wherein said eNOS polypeptide comprises a mutation at a position corresponding to amino acid 1177 of SEQ ID NO: 1.

7. The method according to claim 4, wherein said eNOS polypeptide comprises a first mutation at a position corresponding to amino acid 495 and a second mutation at a position corresponding to amino acid 1177 of SEQ ID NO: 1.

8. The method according to claim 4, wherein said eNOS polypeptide comprises a first mutation at a position corresponding to amino acid 495, a second mutation at a position corresponding to amino acid 1177, and a third mutation at a position corresponding to amino acid 2 of SEQ ID NO: 1.

9. The method according to claim 6, 7, or 8, wherein said mutation at a position corresponding to amino acid residue 495 is an amino acid substitution to Ala, Val, Leu, or Ile.

10. The method according to claim 6, 7, or 8, wherein said mutation at a position corresponding to amino acid residue 1177 is an amino acid substitution to Asp.

11. The method according to claim 8, wherein said mutation at a position corresponding to amino acid residue 2 is an amino acid substitution to Ala.

12. The method according to claim 4, wherein the phosphorylation of said eNOS polypeptide is increased or decreased, as compared to a reference eNOS polypeptide.

13. The method according to claim 4, wherein said eNOS polypeptide has an increased binding affinity for calmodulin, as compared to a reference eNOS polypeptide.

14. The method according to claim 4, wherein Ca++ dependence is decreased in Ca++-calmodulin mediated stimulation of said eNOS polypeptide as compared to a reference eNOS polypeptide.

15. The method according to claim 4, wherein said eNOS polypeptide has increased eNOS activity, as compared to a reference eNOS polypeptide.

16. The method according to claim 15, wherein said activity is the generation of NO.

17. The method according to claim 15, wherein said activity is reductase activity.

18. The method according to claim 12, 13, 14, 15, 16, or 17, wherein the amino acid sequence of said reference polypeptide is, or is derived from, the amino acid sequence of a human eNOS.

19. The method according to claim 18, wherein the amino acid sequence of said reference polypeptide is, or is derived from, SEQ ID NO: 1.

20. The method according to claim 4, wherein the amino acid sequence of said eNOS polypeptide is substantially homologous to the amino acid sequence of a human eNOS.

21. The method according to claim 20, wherein the amino acid sequence of said eNOS polypeptide has a 95-99% sequence identity to the amino acid sequence of SEQ ID NO: 1.

22. The method according to claim 1 or 4, wherein said polynucleotide is a recombinant vector comprising a nucleic acid sequence encoding said eNOS polypeptide and said sequence is operably linked to at least one regulatory sequence such that said polypeptide is expressed in cells.

23. The method according to claim 22, wherein said nucleic acid sequence is operably linked to a promoter.

24. The method according to claim 23, wherein said recombinant vector is a viral vector.

25. The method according to claim 24, wherein said viral vector is an adenoviral vector.

26. The method according to claim 1 or 4, wherein said treating comprises modulating eNOS activity in cells of said patient.

27. The method according to claim 26, wherein said cells are endothelial cells.

28. The method according to claim 26, wherein said cells are bone marrow derived cells.

29. The method according to claim 1 or 4, wherein said method further comprises administering one or more angiogenic factors to said patient, before, during, or after said administering of said polynucleotide.

30. The method according to claim 29, wherein said angiogenic factors are selected from a group of angiogenic factors consisting of: HGF, VEGF, FGF, Endothelial Growth Factor, Epidermal Growth Factor, Platelet-Derived Growth Factor, TGF-alpha, TGF-beta, PDGF, TNA-alpha or IGF, Del-1.

31. The method according to claim 1 or 4, wherein said administering comprises introducing said polynucleotide to cells of said patient ex vivo.

32. The method according to claim 1 or 4, wherein said administering comprises delivery of said polynucleotide to a diseased tissue of said patient.

33. The method according to claim 1 or 4, wherein said administering comprises delivery of said polynucleotide to the peripheral vascular system of said patient.

34. The method according to claim 33, wherein said delivery is by intramuscular injection or intraarterial injection to a limb muscle of said patient.

35. A method of treating angiogenesis comprising administering to a patient in need of treatment an effective amount of a polynucleotide encoding an eNOS polypeptide, wherein said eNOS polypeptide comprises at least one mutation at a position corresponding to an amino acid residue in a mammalian eNOS that is phosphorylated in mammalian cells.

36. A method of ameliorating microvascular dysfunction comprising administering to a patient in need of treatment an effective amount of a polynucleotide encoding an eNOS polypeptide, wherein said eNOS polypeptide comprises at least one mutation at a position corresponding to an amino acid residue in a mammalian eNOS that is phosphorylated in mammalian cells.

37. A method of treating critical limb ischemia (CLI) comprising administering to a patient in need of treatment an effective amount of an eNOS polypeptide, wherein said eNOS polypeptide comprises at least one mutation at a position corresponding to an amino acid residue in a mammalian eNOS that is phosphorylated in mammalian cells.

38. The method according to claim 35, 36, or 37, wherein said eNOS polypeptide comprises a mutation at a position corresponding to amino acid residue 495 of SEQ ID NO: 1, and said mutation is an amino acid substitution to Ala, Val, Leu, or Ile.

39. The method according to claim 35, 36, or 37, wherein said eNOS polypeptide comprises a mutation at a position corresponding to amino acid 1177 of SEQ ID NO: 1, and said mutation is an amino acid substitution to Asp.

40. The method according to claim 1, 35, 36, or 37, wherein said eNOS polypeptide comprises:
i) a first mutation at a position corresponding to amino acid 495, and said first mutation is an amino acid substitution to Ala, Val, Leu, or Ile; and
ii) a second mutation at a position corresponding to amino acid 1177 of SEQ ID NO: 1, and said second mutation is an amino acid substitution to Asp.

41. The method according to claim 1, 35, 36, or 37, wherein said eNOS polypeptide comprises:
i) a first mutation at a position corresponding to amino acid 495, and said first mutation is an amino acid substitution to Ala, Val, Leu, or Ile;
ii) a second mutation at a position corresponding to amino acid 1177 of SEQ ID NO: 1, and said second mutation is an amino acid substitution to Asp; and
iii) a third mutation at a position corresponding to amino acid 2 of SEQ ID NO: 1, and said second mutation is an amino acid substitution to Ala.
